(19) United States
(12) Patent Application Publication
(10) Pub. No.: US 2024/0240204 A1
(43) Pub. Date: Jul. 18, 2024

(54) METHODS AND COMPOSITIONS FOR TRANSPORT, STORAGE, AND DELIVERY OF ADENO-ASSOCIATED VIRAL VECTOR AND OTHER MOLECULES

(71) Applicant: BOARD OF REGENTS, THE UNIVERSITY OF TEXAS SYSTEM, Austin, TX (US)

(72) Inventors: Maria A. CROYLE, Austin, TX (US); Trang DOAN, Austin, TX (US)

(73) Assignee: BOARD OF REGENTS, THE UNIVERSITY OF TEXAS SYSTEM, Austin, TX (US)

(21) Appl. No.: 18/559,460

(22) PCT Filed: May 6, 2022
(86) PCT No.: PCT/US2022/028009

§ 371 Date: Nov. 7, 2023

Related U.S. Application Data
(60) Provisional application No. 63/185,773, filed on May 7, 2021.
(60) Provisional application No. 63/186,626, filed on May 10, 2021.
(60) Provisional application No. 63/187,765, filed on May 12, 2021.
(60) Provisional application No. 63/191,571, filed on May 21, 2021.
(60) Provisional application No. 63/300,970, filed on Jan. 19, 2022.

(51) Int. Cl. C12N 15/86; A61K 9/70; A61K 47/26; A61K 47/34; A61K 47/38

(52) U.S. Cl. CPC C12N 15/86; A61K 9/7007; A61K 47/26; A61K 47/34; A61K 47/38; C12N 2750/14143; C12N 2750/14171

(57) ABSTRACT

Aspects of the present disclosure are directed to liquid and substantially solid film compositions for storage, transport, and administration of parvovirus and parvovirus vectors, including adeno-associated vims (AAV) vectors, without the use of ultralow temperatures. Provided are compositions capable of long-term storage of AAV vectors at ambient temperatures. Also disclosed are injectable compositions comprising AAV vectors, as well as methods for formulation and administration of such compositions.

### CROSS REFERENCE TO RELATED APPLICATIONS

This application claims benefit of priority to U.S. Provisional Application No. 63/185,773, filed Mar. 7, 2021, U.S. Provisional Application No. 63/186,626, filed May 10, 2021, U.S. Provisional Application No. 63/187,765, filed May 12, 2021, U.S. Provisional Application No. 63/191,571, filed May 21, 2021, and U.S. Provisional Application No. 63/300,970, filed Jan. 19, 2022, all of which are hereby incorporated by reference in their entirety.

### BACKGROUND

### I. Field of the Invention

Aspects of the invention relate to at least the fields of biotechnology and pharmaceutical chemistry. More particularly, aspects relate to compositions and methods for storage, preservation, and delivery of biological materials, including virus vectors such as adeno-associated virus vectors.

### II. Background

Recombinant adeno-associated viruses (AAVs) have been developed as vectors for gene therapy. AAVs are under evaluation as therapies for various conditions, including monogenetic, ocular, cardiovascular, lysosomal storage, neuromuscular and infectious diseases. Breakthroughs in recombinant DNA and bioprocessing technologies have allowed AAV to be among the first viral vectors to gain regulatory approval by the United States Food and Drug Administration (FDA). However, current AAV products are formulated as liquid products that are stored and shipped at ultralow temperatures. This approach poses significant logistic and economic issues with respect to global distribution and access to these life-saving medicines.

Accordingly, recognized is a need for compositions and methods for storage, maintenance, and delivery (e.g., via intravenous delivery) of viral vectors such as AAV vectors without the need for ultralow temperatures.

### SUMMARY

Aspects of the present disclosure address certain needs in the art by providing liquid and film compositions capable of long-term storage of adeno-associated virus (AAV), and other parvovirus vectors at temperatures above freezing, including refrigeration (e.g., 4° C.) and ambient temperatures (e.g., 25° C.), while retaining high efficacy and infectivity. Accordingly, certain aspects are directed to compositions comprising an AAV vector in a carrier comprising a zwitterionic surfactant (e.g., PMAL such as PMAL-C16) and hydroxypropyl methylcellulose (HPMC). As disclosed herein, in some cases, compositions comprising HPMC having particular molecular weight and/or viscosity may be particularly conducive for generation of stable, injectable formulations. Thus, aspects of the disclosure include compositions comprising an agent in a substantially solid carrier comprising hydroxypropyl methylcellulose (HPMC) and a zwitterionic surfactant, wherein the HPMC has a molecular weight (MW) that produces a viscosity that is less than 4000 cp (e.g., less than 3000, 2000, 1800, 500, 300, or 200 cp) at a concentration of 2% in water. As disclosed herein, HPMC viscosity is described in terms of viscosity at a concentration of the HPMC of 2% in water, unless otherwise indicated. Also disclosed are methods for generating such compositions, methods for storage of such compositions, and methods of administration of such compositions, for example administration to a patient for therapeutic purposes including gene therapy.

Disclosed herein, in some aspects, is a composition comprising an AAV vector in a carrier comprising (a) a zwitterionic surfactant and (b) hydroxypropyl methylcellulose (HPMC). In some aspects, the HPMC is between 0.5% and 3.0%, for example 0.6, 0.7, 0.8, 0.9, 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, or 3% HPMC, or any range or value derivable therein. In certain aspects, the HPMC has a molecular weight that produces a viscosity that is less than 4000, 3500, 3000, 2500, 2000, 1800, 1500, 1200, 1000, 800, 600, 500, 400, 300, 200, 100, 50, 20, 15, 14, 13, 12, 11, 10, 9, 8, 7, 6, or 5 centipoise (cp) at a concentration of 2% in water, including any range or value derivable therein. In some aspects, the carrier comprises between 0.5% and 5.0% of a sugar, for example 0.5, 0.6, 0.7, 0.8, 0.9, 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3, 3.1, 3.2, 3.3, 3.4, 3.5, 3.6, 3.7, 3.8, 3.9, 4, 4.1, 4.2, 4.3, 4.4, 4.5, 4.6, 4.7, 4.8, 4.9, or 5% of a sugar, including any range or value derivable therein. Various sugars are disclosed herein and include, for example, glucose, dextrose, fructose, lactose, maltose, xylose, sucrose, corn sugar syrup, sorbitol, hexitol, maltilol, xylitol, mannitol, melezitose, and raffinose. Any one of these sugars may be excluded from a composition of the disclosure in certain aspects. In some aspects, the composition further comprises between 0.5% and 5.0% of glycerol, for example 0.5, 0.6, 0.7, 0.8, 0.9, 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3, 3.1, 3.2, 3.3, 3.4, 3.5, 3.6, 3.7, 3.8, 3.9, 4, 4.1, 4.2, 4.3, 4.4, 4.5, 4.6, 4.7, 4.8, 4.9, or 5% of glycerol. In some aspects, the zwitterionic surfactant is PMAL-C16, where in some cases the carrier comprises between 0.1% and 5% PMAL-C16, for example 0.1, 0.2, 0.3, 04, 0.5, 0.6, 0.7, 0.8, 0.9, 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3, 3.1, 3.2, 3.3, 3.4, 3.5, 3.6, 3.7, 3.8, 3.9, 4, 4.1, 4.2, 4.3, 4.4, 4.5, 4.6, 4.7, 4.8, 4.9, or 5% PMAL-C16. In some aspects, a composition of the disclosure is a liquid, in some aspects, a composition of the disclosure is a substantially solid film.

Further disclosed are methods for storing an AAV vector comprising formulating the AAV vector in a composition of the disclosure and storing the composition, for example storing the composition at temperatures above 0° C., above 15° C., above 25° C., above 30° C., or above 35° C., for at least 7 days, at least 14 days, at least 30 days, at least 60 days, or at least 90 days. Also disclosed are methods for delivering an AAV vector to a subject comprising administering to the subject an effective amount of a composition of the present disclosure, in some cases where the composition has been stored (e.g., for at least 7, 14, 30, or more days) at a temperature above 0° C. (e.g., about 4° C., about 25° C., between 15-30° C., or between 0-8° C.) prior to administration to the subject.

Disclosed, in some aspects, is a method for making a stabilized AAV vector composition, the method comprising forming an aqueous solution comprising an AAV vector, a zwitterionic surfactant, and HPMC. In some aspects, the method further comprises drying the aqueous solution to form a substantially solid film. In some aspects, the method does not comprise drying the aqueous solution to form a substantially solid film. In some aspects, the method further comprises dissolving the substantially solid film in an appropriately buffered aqueous solution to generate the pharmaceutical composition in liquid reconstituted form.

Aspects of the invention relate to a pharmaceutical composition comprising between 1×10⁶ to about 1×10¹⁶ vg/ml of an AAV vector formulated within from about 0.1% to about 5% wt/vol hydroxypropyl methylcellulose (HPMC); from about 0.5% to about 5% glycerol; from about 0.5% to about 5% sorbitol; and from about 0.1% to about 5% PMAL-C16. In certain aspects, the pH of one or more of the components of the composition is adjusted so that the final pH of the composition is from about pH 6.0 to 8.0. In some aspects of the composition and methods disclosed herein, the HPMC is 1.5%, the glycerol is 2%, the sorbitol is 2% and the PMAL-C16 is 1%. In some aspects of the composition and methods disclosed herein, the composition is in liquid form. In some aspects of the composition and methods disclosed herein, the composition is in substantially solid film form. In some aspects of the composition and methods disclosed herein, the pH is adjusted so that the final pH of the composition is from about pH 6.5 to about pH 8.5. In some aspects of the composition and methods disclosed herein, the pH is adjusted so that the final pH of the composition is about 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, or 8.5. In some aspects of the composition and methods disclosed herein, the composition is generated by the following steps of combining the HPMC in aqueous form with the glycerol, sorbitol and PMAL-C16, in the presence of a buffering agent sufficient to promote from pH 6.0 to 8.5 of the composition, to thereby generate a homogeneous mixture; and adding viral vector to the homogeneous mixture at ambient temperature to disperse therein to thereby generate the pharmaceutical composition in liquid form. In some aspects of the composition and methods disclosed herein, the steps further include drying the pharmaceutical composition under ambient temperature and pressure to thereby form a substantially solid film. In some aspects of the composition and methods disclosed herein, the steps further include dissolving the substantially solid film in an appropriately buffered aqueous solution to generate the pharmaceutical composition in liquid reconstituted form.

In some aspects of the composition and methods disclosed herein, the liquid pharmaceutical composition is stored for at least 30 days, at about 4° C., about 25° C., between 0° C. and 40° C., is subject to freeze thawing, or combinations thereof, prior to drying step and/or wherein the substantially solid film is stored for at least 30 days, at about 4° C., about 25° C., between 0° C. and 40° C., is subject to freeze thawing, or combinations thereof, prior to dissolving step to generate the liquid reconstituted form. In some aspects of the composition and methods disclosed herein, the film is stored for 150 or more days. In some aspects of the composition and methods disclosed herein, the film is stored for at least 6 months, at least 12 months, at least 18 months, at least 24 months, or at least 36 months.

In some aspects of the composition and methods disclosed herein, the AAV vector is preserved by at least 40% as measured by infectivity, transduction efficiency, and/or vector genome copy, number following storage. In some aspects of the composition and methods disclosed herein, the AAV vector is preserved by at least 45% as measured by infectivity, transduction efficiency, and/or vector genome copy, number following storage. It is believed that considerable value is obtained even with a composition described herein that results in 45% or less (e.g., about 40%, about 35%, about 30%, about 25%, etc.) preservation of AAV vector activity, in light of the ease of storage and shipment.

In some aspects of the composition and methods disclosed herein, the AAV vector is preserved by at least 50% as measured by infectivity, transduction efficiency, and/or vector genome copy, number following storage. In some aspects of the composition and methods disclosed herein, the AAV vector is preserved by at least 55% as measured by infectivity, transduction efficiency, and/or vector genome copy, number following storage. In some aspects of the composition and methods disclosed herein, the AAV vector is preserved by at least 60% as measured by infectivity, transduction efficiency, and/or vector genome copy, number following storage. In some aspects of the composition and methods disclosed herein, the AAV vector is preserved by at least 65% as measured by infectivity, transduction efficiency, and/or vector genome copy, number following storage. In some aspects of the composition and methods disclosed herein, the AAV vector is preserved by at least 70% as measured by infectivity, transduction efficiency, and/or vector genome copy, number following storage. In some aspects of the composition and methods disclosed herein, the AAV vector is preserved by at least 75% as measured by infectivity, transduction efficiency, and/or vector genome copy, number following storage. In some aspects of the composition and methods disclosed herein, the AAV vector is preserved by at least 80% as measured by infectivity, transduction efficiency, and/or vector genome copy, number following storage. In some aspects of the composition and methods disclosed herein, the AAV vector is preserved by at least 85% as measured by infectivity, transduction efficiency, and/or vector genome copy, number following storage. In some aspects of the composition and methods disclosed herein, the AAV vector is preserved by at least 90% as measured by infectivity, transduction efficiency, and/or vector genome copy, number following storage. In some aspects of the composition and methods disclosed herein, the AAV vector is preserved by at least 95% as measured by infectivity, transduction efficiency, and/or vector genome copy, number following storage. In some aspects of the composition and methods disclosed herein, the AAV vector is preserved by at least 99% as measured by infectivity, transduction efficiency, and/or vector genome copy, number following storage.

In some aspects of the composition and methods disclosed herein, the HPMC and/or PMAL-C16 are from about 0.5% to about 3.0%, or from about 1.0% to about 2.0%, or from about 1% to about 1.5%, or about 0.5%, about 1%, about 1.5%, about 2%, about 2.5%, or about 3%.

In some aspects of the composition and methods disclosed herein, the sorbitol and/or glycerol are from about 0.5% to about 5.0%, or from about 1.0% to about 4.0%, or from about 1%-3%, or about 0.5%, about 1%, about 1.5%, about 2%, about 2.5%, about 3%, about 3.5%, about 4%, about 4.5%, or about 5%.

In some aspects of the composition and methods disclosed herein, the HPMC has a MW that produces a viscosity of from about 12 cp to about 4000 cp at a concentration of 2% in water.

In some aspects of the composition and methods disclosed herein, the HPMC has a MW that produces a viscosity of less than 4000 cp, less than 3000 cp, less than 2000 cp, less than 1800 cp, or less than 400 cp at a concentration of 2% in water. In some aspects of the composition and methods disclosed herein, the HPMC has a MW that produces a viscosity of from about 12 cp to about 3000 cp, or from about 12 cp to about 1800 cp at a concentration of 2% in water.

In some aspects of the composition and methods disclosed herein, the HPMC has a MW that produces a viscosity of from about 12 cp to about 400 cp, from about 12 cp to about 100 cp, or from about 100 cp to about 400 cp at a concentration of 2% in water.

The use of a combination of various molecular weight HPMC is also envisioned. In some aspects of the composition and methods disclosed herein, the HPMC is A4M, F4M, A15C, A4C, K100LV, E4M, E6LV, or A15LV. A composition of the disclosure may exclude one or more particular molecular weight HPMC in certain aspects. In some aspects, the HPMC is not K4M.

In some aspects of the composition and methods disclosed herein, the AAV vector is present from about 1×10⁶ to about 1×10¹⁶ vg/ml volume. In some aspects of the composition and methods disclosed herein, the AAV vector is present in between 1×10⁶ to about 1×10¹⁶/film generated from 1 ml liquid volume.

In some aspects of the composition and methods disclosed herein, the AAV vector comprises a capsid that is selected from an AAV1 vector, an AAV2 vector, an AAV3 vector, an AAV4 vector, an AAV5 vector, an AAV6 vector, an AAV7 vector, an AAV8 vector, an AAV9 vector, an AAV10 vector, an AAV11 vector, an AAV12 vector, an AAV13 vector, and hybrids thereof. In some aspects, the AAV vector is an AAV1 vector, an AAV2 vector, an AAV3 vector, an AAV4 vector, an AAV5 vector, an AAV6 vector, an AAV7 vector, an AAV8 vector, an AAV9 vector, an AAV10 vector, an AAV11 vector, an AAV12 vector, an AAV13 vector, or a hybrid thereof. In some aspects, the AAV vector is an AAV9 vector.

In some aspects of the composition and methods disclosed herein, the AAV vector comprises an expression cassette (e.g., therapeutic or diagnostic agent, e.g., polypeptide or nucleic acid), an AAV vector of the present disclosure include any AAV vector comprising an expression cassette for any molecule, nucleic acid (e.g., RNA), peptide, polypeptide, or other agent. In certain aspects, an AAV vector of the present disclosure is an AAV vector encoding for an antibody, for example a therapeutic antibody.

In some aspects of the compositions and methods disclosed herein, the composition is a liquid or substantially solid amorphous carrier.

Aspects of the invention relate to a pharmaceutical composition generated by combining the composition described above and a pharmaceutically acceptable carrier. (e.g., to dilute the composition and/or further prepare for administration to a subject).

Aspects of the invention relate to a method of storing/preserving an AAV vector comprising, formulating the AAV vector in a composition described herein.

Aspects of the invention relate to a method of storing an AAV vector, for at least 120 days, at a temperature between 0° C.-40° C. comprising using the pharmaceutical composition described herein.

In some aspects of the methods described herein, the method comprises generating the composition by the following steps, combining the HPMC in aqueous form with the glycerol, sorbitol and PMAL-C16, in the presence of a buffering agent sufficient to promote from pH 6.0 to 8.5 of composition, to thereby generate a homogeneous mixture; adding AAV vector to the homogeneous mixture at ambient temperature to disperse therein to thereby generate the pharmaceutical composition in liquid form.

In some aspects of the methods described herein, the AAV vector is stored for at least 150 days, at least 6 months, at least 12 months, at least 18 months, at least 24 months, or at least 36 months, or more.

In some aspects of the methods described herein, the composition is stored for at least 30 days, at about 4° C., about 0° C., about 25° C., is subject to freeze thawing, or combinations thereof.

In some aspects of the methods described herein, the composition is stored for 150 or more days. In some aspects of the methods described herein, the composition is stored for at least, at most, about, or exactly 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 45, 60, 90, 120, or 150 days, including any range or value derivable therein.

In some aspects of the methods described herein, the composition is stored for at least 6 months, at least 12 months, at least 18 months, at least 24 months, or at least 36 months, or more.

In some aspects of the methods described herein, the composition preserved the AAV vector therein by at least 40%, as measured by infectivity, transduction efficiency, and/or vector genome copy, number following storage. In some aspects, the AAV vector is preserved by at least 45% as measured by infectivity, transduction efficiency, and/or vector genome copy, number following storage. In some aspects, the AAV vector is preserved by at least 50% as measured by infectivity, transduction efficiency, and/or vector genome copy, number following storage. In some aspects, the AAV vector is preserved by at least 55% as measured by infectivity, transduction efficiency, and/or vector genome copy, number following storage. In some aspects, the AAV vector is preserved by at least 60% as measured by infectivity, transduction efficiency, and/or vector genome copy, number following storage. In some aspects, the AAV vector is preserved by at least 65% as measured by infectivity, transduction efficiency, and/or vector genome copy, number following storage. In some aspects, the AAV vector is preserved by at least 70% as measured by infectivity, transduction efficiency, and/or vector genome copy, number following storage. In some aspects, the AAV vector is preserved by at least 75% as measured by infectivity, transduction efficiency, and/or vector genome copy, number following storage. In some aspects, the AAV vector is preserved by at least 80% as measured by infectivity, transduction efficiency, and/or vector genome copy, number following storage. In some aspects, the AAV vector is preserved by at least 85% as measured by infectivity, transduction efficiency, and/or vector genome copy, number following storage. In some aspects, the AAV vector is preserved by at least 90% as measured by infectivity, transduction efficiency, and/or vector genome copy, number following storage. In some aspects, the AAV vector is preserved by at least 95% as measured by infectivity, transduction efficiency, and/or vector genome copy, number following storage. In some aspects, the AAV vector is preserved by at least 99% as measured by infectivity, transduction efficiency, and/or vector genome copy, number following storage.

Aspects of the disclosure relate to a method of delivering an AAV vector to a subject comprising administering to the subject an effective amount of the compositions described herein. In some aspects, the method further comprises dispersing the composition in a pharmaceutically acceptable carrier. In some aspects, the composition is administered intravenously, intradermally, intra-arterially, intra-graft, intraperitoneally, intralesionally, intracranially, intraspinally, intracisternally, intraarticularly, intraprostatically, intrapleurally, intratracheally, intranasally, intravitreally, intravaginally, intrarectally, topically, intratumorally, intramuscularly, intraperitoneally, subcutaneously, subconjunctivally, intravesicularly, mucosally, intrapericardially, intraumbilically, intraocularly, orally, topically, locally, inhalation, injection, infusion, continuous infusion, localized perfusion bathing target cells directly, via a catheter, via lavage, in cremes, in lipid compositions (e.g., liposomes), or combinations thereof. In particular aspects, disclosed is a method of delivering an AAV vector to a subject comprising intravenously administering an effective amount of a composition described herein.

Aspect 1 includes a composition comprising an adeno-associated virus (AAV) vector in a carrier comprising (a) a zwitterionic surfactant and (b) hydroxypropyl methylcellulose (HPMC). Aspect 2 depends on aspect 1, wherein the HPMC is between 0.5% and 3.0%. Aspect 3 depends on aspect 1, wherein the HPMC is about 1.5%. Aspect 4 depends on aspects 1 or 2, wherein the HPMC has a molecular weight (MW) that produces a viscosity that is less than 4000 cp at a concentration of 2% in water. Aspect 5 depends on aspects 1 or 2, wherein the HPMC is A4M, F4M, A15C, A4C, K100LV, E4M, E6LV, or A15LV. Aspect 6 depends on any of aspects 1-5, wherein the carrier further comprises a sugar. Aspect 7 depends on any of aspects 1-5, wherein the carrier comprises about 2.0% sorbitol. Aspect 8 depends on any of aspects 1-7, wherein the carrier comprises about 2.0% glycerol. Aspect 9 depends on any of aspects 1-8, wherein the carrier comprises between 0.1% and 5% of the zwitterionic surfactant. Aspect 10 depends on aspect 9, wherein the carrier comprises about 1% PMAL-C16. Aspect 11 depends on any of aspects 1-10, wherein the composition has a pH from 7.0 to 9.0. Aspect 12 depends on any aspects 1-11, wherein the carrier comprises about 1.5% HPMC, about 2% glycerol, about 2% sorbitol, and about 1% PMAL-C16. Aspect 13 depends on any of aspects 1-12, wherein the composition is a liquid. Aspect 14 depends on any of aspects 1-12, wherein the composition is a substantially solid film. Aspect 15 includes a method for storing an AAV vector comprising formulating the AAV vector in a composition of any of aspects 1-14. Aspect 16 depends on aspect 15, wherein the method comprises storing the AAV vector in the composition for at least 30 days at a temperature of at least 0° C. Aspect 17 depends on aspects 15 or 16, wherein, after storing, the AAV vector is preserved by at least 80% as measured by infectivity, transduction efficiency, and/or vector genome copy. Aspect 18 includes a method of delivering an AAV vector to a subject, the method comprising administering to the subject an effective amount of the composition of any of claim 1-14. Aspect 19 depends on aspect 18, the composition is administered to the subject intravenously. Aspect 20 depends on aspect 18 or 19, further comprising, prior to administering the composition to the subject, storing the composition for at least 30 days at a temperature of at least 0° C.

Aspect 21 includes a method for making a stabilized AAV vector composition, the method comprising forming an aqueous solution comprising an AAV vector, a zwitterionic surfactant, and HPMC. Aspect 22 depends on aspect 21, wherein the HPMC has a molecular weight (MW) that produces a viscosity that is less than 4000 cp at a concentration of 2% in water. Aspect 23 depends on aspect 22, wherein the aqueous solution comprises about 1.5% HPMC, about 2% glycerol, about 2% sorbitol, and about 1% PMAL-C16, wherein the aqeuous solution has a pH between 7.0 and 9.0. Aspect 24 depends on any of aspects 21-23, further comprising drying the aqueous solution to form a substantially solid film. Aspect 24 depends on aspect 24, further comprising (a) storing the substantially solid film for at least 30 days at a temperature of at least 0° C.; and (b) dissolving the substantially solid film in an appropriately buffered aqueous solution.

Aspect 26 includes a pharmaceutical composition comprising between 1×10⁶ to about 1×10¹⁶ vg/ml of an AAV vector formulated within (a) from about 0.1% to about 5% wt/vol hydroxypropyl methylcellulose (HPMC) having a molecular weight (MW) that produces a viscosity that is less than 4000 cp in 2% water; (b) from about 0.5% to about 5% glycerol; (c) from about 0.5% to about 5% sorbitol; and (d) from about 0.1% to about 5% PMAL-C16; and wherein the pH of the composition is from about pH 6.0 to 9.0. Aspect 27 depends on aspect 26, wherein the composition comprises 1.5% HPMC, 2% glycerol, 2% sorbitol, 1% PMAL-C16, and has a pH of between 7.0 and 9.0.

Aspect 28 includes a composition comprising an agent in a substantially solid carrier comprising hydroxypropyl methylcellulose (HPMC) and a zwitterionic surfactant, wherein the HPMC has a molecular weight (MW) that produces a viscosity that is less than 4000 cp at a concentration of 2% in water, and wherein the composition has a pH from 7.0 to 9.0. Aspect 29 depends on aspect 28, wherein the HPMC has a MW that produces a viscosity that is less than 1800 cp at a concentration of 2% in water. Aspect 30 depends on aspect 28 or 29, wherein the agent is an adeno-associated virus (AAV) vector. Aspect 31 depends on aspect 28 or 29, wherein the agent is a polypeptide, small molecule, or nucleic acid. Aspect 32 depends on any of aspects 28-31, wherein the carrier comprises about 1.5% HPMC, about 2% glycerol, about 2% sorbitol, and about 1% PMAL-C16.

The term “about” or “approximately” are defined as being close to as understood by one of ordinary skill in the art. In one non-limiting embodiment the terms are defined to be within 10%, preferably within 5%, more preferably within 1%, and most preferably within 0.5%.

The use of the word “a” or “an” when used in conjunction with the term “comprising” may mean “one,” but it is also consistent with the meaning of “one or more,” “at least one,” and “one or more than one.”

The phrase “and/or” means “and” or “or”. To illustrate, A, B, and/or C includes: A alone, B alone, C alone, a combination of A and B, a combination of A and C, a combination of B and C, or a combination of A, B, and C. In other words, “and/or” operates as an inclusive or.

The words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The compositions and methods for their use can “comprise,” “consist essentially of,” or “consist of” any of the ingredients or steps disclosed throughout the specification. Compositions and methods “consisting essentially of” any of the ingredients or steps disclosed limits the scope of the claim to the specified materials or steps which do not materially affect the basic and novel characteristic of the claimed invention.

“Individual,” “subject,” and “patient” are used interchangeably and can refer to a human or non-human.

“Ambient temperature” and “room temperature” can each include a temperature of 15° C. to 30° C., or any range or value derivable therein. In some aspects, ambient or room temperature can include a temperature of 15° C. to 25° C., 20° C. to 25° C., 18° C. to 28° C., or any range or value derivable therein. In certain aspects, ambient or room temperature includes 25° C.

Any method in the context of a therapeutic, diagnostic, or physiologic purpose or effect may also be described in “use” claim language such as “Use of” any compound, composition, or agent discussed herein for achieving or implementing a described therapeutic, diagnostic, or physiologic purpose or effect.

It is specifically contemplated that any limitation discussed with respect to one embodiment of the invention may apply to any other embodiment of the invention.

Furthermore, any composition of the invention may be used in any method of the invention, and any method of the invention may be used to produce or to utilize any composition of the invention. Any embodiment discussed with respect to one aspect of the disclosure applies to other aspects of the disclosure as well and vice versa. For example, any step in a method described herein can apply to any other method. Moreover, any method described herein may have an exclusion of any step or combination of steps. Aspects of an embodiment set forth in the Examples are also embodiments that may be implemented in the context of embodiments discussed elsewhere in a different Example or elsewhere in the application, such as in the Summary, Detailed Description, Claims, and Brief Description of the Drawings.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIGS. 1A-1D show stability profile of recombinant AAV for 48 hours and 7 days at 4 and 25° C. prior to reformulation in film matrix. Data from freshly purified virus stored at −80° C. is included as a point of reference. AAV expressing the luciferase transgene was formulated and replicate vials placed at 4 and 25° C. Three vials per timepoint were collected and assayed for transgene expression by serial dilution on HeLa RC32 cells and luciferase expression assessed 72 hours later (FIGS. 1A and 1B). Replicate plates of cells were harvested and virus genome copies assessed by quantitative real time PCR (FIGS. 1C and 1D). ***p<0.001, one way ANOVA with Dunnett's post-hoc test.

FIGS. 2A-2E show results demonstrating that pH and film components significantly impact recovery of active virus from film matrix. Polymer base compositions with decreasing viscosities were screened for their film-forming capacity and preservation of virus activity during drying (FIG. 2A). Recovery of AAV from films prepared with polymer base buffered to different pH after drying was evaluated using an infectious titer assay (FIG. 2B). The impact of the pH of the polymer base was more evident after films were stored at room temperature for 14 days (FIG. 2C). Additional screening revealed that a surfactant significantly improved recovery of live virus from the film matrix (FIG. 2E) in a size dependent manner (FIG. 2D). Data represents the average ± the standard error of the mean of 3 films per condition. *p<0.05, **p<0.01, ***p<0.001, one way ANOVA with Tukey's multiple comparison tests.

FIGS. 3A-3C show results demonstrating that viscosity of polymer matrix significantly impacts release profile of virus from film. FIG. 3A shows cumulative release profile of films prepared with high (F1S) and low (F2S) viscosity polymers as determined by transgene expression. Films containing 1×10¹² virus genomes were placed in warmed (37° C.) phosphate buffered saline with gentle agitation and samples collected over a period of 2 hours. Infectious titer of virus released in was determined by infection of RC32 cells and assessment of luciferase expression. FIG. 3B shows cumulative release profile of virus genomes from films as determined by quantitative real time PCR. Curves followed a similar trend as those based upon transgene expression. FIG. 3C shows average release rate of AAV Genomes from film formulations. Data collected during the dissolution of each film was normalized with that generated from virus placed in the correlating liquid formulations to account for any loss attributable to agitation and extended exposure to heat. In each panel, data represents the average ± the standard error of the mean of five films per condition. ***p<0.001, two-tailed Student's t test.

FIGS. 4A and 4B show results demonstrating that formulation and environmental humidity significantly impact residual moisture content and long term stability of AAV within the film matrix. FIG. 4A shows residual moisture in high (F1S) and low (F2S) viscosity films after drying. Data represents the average± the standard error of the mean for 3 replicates for each formulation. **p<0.01, two-tailed Student's t test. FIG. 4B shows results demonstrating that sixty percent relative humidity maintains AAV Stability within the film matrix. Films containing 1×10¹² virus genomes were prepared in batch of the high viscosity formulation and stored at 25° C. under different humidity environments. Films (3 per timepoint, and storage condition) were rehydrated with warm culture media and infectious titer determined by limiting dilution. *p<0.05, **p<0.01, ***p<0.001, two way ANOVA with Tukey's post-hoc multiple comparison tests.

FIGS. 5A-5C show results demonstrating that optimized film matrices significantly improve AAV stability. Films containing 1×10¹² virus genomes were prepared in batch and either stored in controlled environmental chambers held at 4° C./40-50% RH (FIG. 5A) or 25° C./60% RH (FIG. 5B) or subjected to a series of 16 freeze-thaw cycles (FIG. 5C). Replicates (at least 3 per timepoint) were reconstituted and live virus concentration assessed by a standard infectious titer assay. In FIGS. 5A and 5B, Control Formulation (OF) consisted of phosphate buffered saline, 350 mM NaCl, 5% Sorbitol, 0.001% Pluronic F68 (pH 7.1). In each panel, data represents the average ± the standard error of the mean of 3 replicates per timepoint or condition. In FIGS. 5A and 5B, significant differences between subgroups with respect to the Control (OF) formulation were evaluated by two way ANOVA with Dunnett's post-hoc tests *p<0.05, **p<0.01, ***p<0.001.

FIGS. 6A-6C show in vivo performance of AAV stabilized in thin film for 30 days at 4° C. Mice were given 1.5×10¹¹ virus genomes of formulated AAV9 CBA-Luc by tail vein injection. Thirty days after administration, animals were sacrificed, organs dissected, processed and tested for luciferase transgene expression with bioluminescence imaging (FIG. 6A). Mean (±standard error of the mean) values for total flux for each organ listed are shown. Organs with high transgene expression were further analyzed for virus genome copies by quantitative real time PCR (FIG. 6B). Data reflect the mean (±standard error of the mean) values for each treatment group. Significant differences between subgroups were evaluated by one way ANOVA with Dunnetts's multiple comparison tests. *p<0.05 Bioluminescent images of mice from each treatment group are shown at 1, 8, 15, 22 and 29 days (FIG. 6C). Relative bioluminescence intensity is shown in pseudo-color, with red and blue representing the strongest and weakest photon fluxes, respectively. Treatment Groups: Vehicle (saline control, Group 1 FIG. 6C); FFF Freshly Purified Virus in Original Frozen Formulation (Group 2 FIG. 6C); RT, FFF Freshly Purified Virus in Original Frozen Formulation held at room temperature (RT) for time taken for film formation to be complete (Group 3 FIG. 6C); F1S (High Viscosity Film Formulation, Group 4 FIG. 6C); F2S (Low Viscosity Film Formulation, Group 5 FIG. 6C). Abbreviations: Diaph.: Diaphragm, Gastroc.: Gastrocnemius muscle.

FIGS. 7A and 7B show results demonstrating that AAV stabilized in thin film for 150 days at 4° C. performs in vivo in a dose dependent manner equivalent to that of frozen vector. Films prepared with high (F1S) and low (F2S) viscosity formulations were rehydrated and diluted with saline to administer 1×10¹⁰ (Dose 1) or 1×10¹¹ (Dose 2) virus genomes of AAV9 CBA-Luc by tail vein injection. Thirty days after administration, animals were sacrificed, organs dissected, processed and tested for luciferase transgene expression with bioluminescence imaging (FIG. 7A). Mean (±standard error of the mean) values for total flux for each organ listed are shown. Significant differences between subgroups were evaluated by one way ANOVA with Dunnett's multiple comparison tests. Organs with high transgene expression were further analyzed for virus genome copies by quantitative real time PCR (FIG. 7B). Data reflect the mean (±standard error of the mean) values for each treatment group. Significant differences between subgroups were evaluated by one way ANOVA with Tukey's multiple comparison tests. Abbreviations: Diaph.: Diaphragm, Gastroc.: Gastrocnemius muscle *p<0.05, **p<0.01.

FIGS. 8A and 8B show results demonstrating that AAV stabilized in thin film for 100 days at room temperature induces transgene expression in a dose dependent manner. Freshly prepared and aged films created with the high viscosity formulation were rehydrated and diluted with saline to administer 1×10¹⁰ (Dose 1) or 1×10¹¹ (Dose 2) virus genomes of AAV9 CBA-Luc by tail vein injection. Thirty days after administration, animals were sacrificed, organs dissected, processed and tested for luciferase transgene expression with bioluminescence imaging (FIG. 8A). Mean (±standard error of the mean) values for total flux for each organ listed are shown. Significant differences between subgroups were evaluated by one way ANOVA with Dunnett's multiple comparison tests. Organs with high transgene expression were further analyzed for virus genome copies by quantitative real time PCR (FIG. 8B). Data reflect the mean (±standard error of the mean) values for each treatment group. Significant differences between subgroups were evaluated by one way ANOVA with Dunnett's multiple comparison tests. Abbreviations: Diaph.: Diaphragm, Gastroc.: Gastrocnemius muscle *p<0.05, **p<0.01, ***p<0.001.

FIG. 9 shows data demonstrating that AAV particles are tightly bound to components of the film matrix. Samples containing 1.67×10¹¹ vg of AAV9 were loaded into well of a 10% polyacrylamide gel. Proteins were resolved by electrophoresis at 80V for 30 minutes followed by 120V for an additional 90 minutes. Protein bands were developed by silver staining using the PlusOne Silver Staining Kit (GE Health Science, Uppsala, Sweden). Samples in lanes from L to R were obtained from aliquots of: A. Rehydrated Placebo Film (containing no virus), B. Rehydrated Film Containing AAV, C. Rehydrated Placebo Film after extensive boiling, D. Rehydrated Placebo Film containing AAV after extensive boiling. E. Freshly Purified AAV, F. AAV after two freeze-thaw cycles, G. AAV after one freeze-thaw cycle, H. Precision Plus Protein™ Dual Color molecular weight standards (10-250 kDa, Bio-Rad, Hercules, CA).

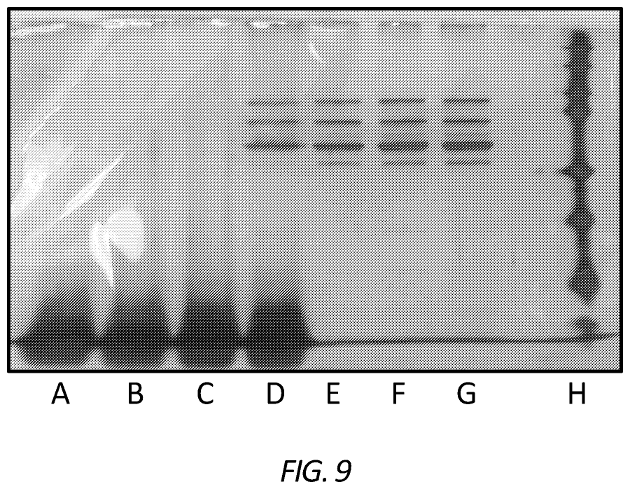

FIGS. 10A and 10B show cytotoxicity profile of AAV in the presence of individual components and composite film formulations (FIG. 10A) and with different doses of AAV9 tested in vivo in the full formulation (FIG. 10B). HelaRC32 cells were treated for a period of 2 hours with each preparation containing AAV (1×10¹¹ vg Panel A, specified amounts FIG. 10B) and cell viability assessed by measuring the amount of active adenosine triphosphate (ATP) present using a Cell Titer-Glo Luminescent Cell Viability assay (Promega). Abbreviations: FIG. 10A. OFF: Original Fresh Frozen Formulation, Buff: 10 mM Tris buffer pH 8.1, P1: high viscosity polymer base, P2: low viscosity polymer base, S: sugar, SF: surfactant. FIG. 10B. F1S: high viscosity complete formulation, F2S: low viscosity complete formulation. Both FIGS. 10A and 10B. Con: Media treated control. Results obtained from each treated group were normalized to results obtained from this group representing a fully viable cell population. ***p<0.001.

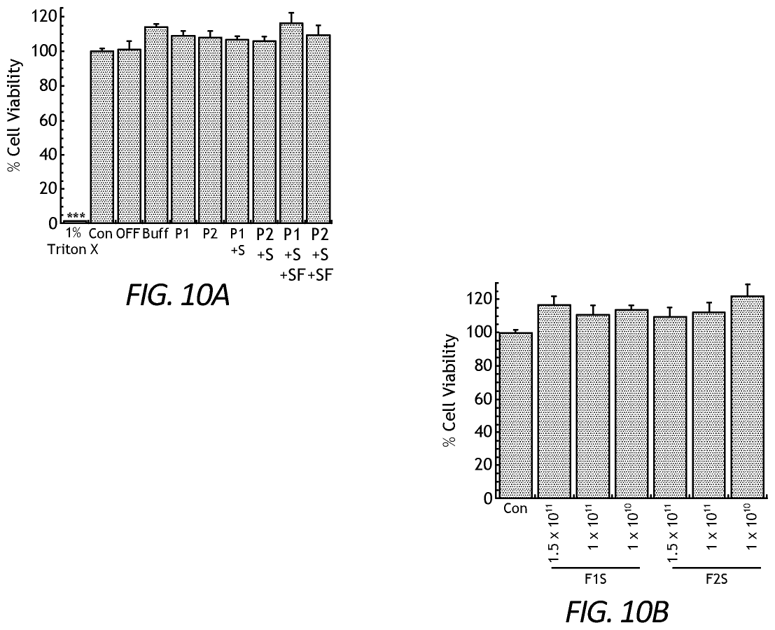

FIG. 11 shows infectious titer of AAV stored in the shown formulations, as described in Example 9. Data represent the averages of 5 individual samples at each timepoint.

FIG. 12 shows infectious titer of AAV stored in the shown formulations, as described in Example 9. Data represent the averages of 5 individual samples at each timepoint.

FIG. 13 shows infectious titer of AAV stored in the shown formulations, as described in Example 9. Data represent the averages of 5 individual samples at each timepoint

FIG. 14 shows infectious titer of AAV stored in the shown formulations, as described in Example 9. Data represent the averages of 5 individual samples at each timepoint

### DETAILED DESCRIPTION OF THE INVENTION

Aspects of the present disclosure relate generally to compositions comprising parvovirus vectors, including adeno-associated virus (AAV) vectors, formulated in stabilizing carriers, including liquid carriers and substantially solid carriers such as a thin film matrix. Also disclosed are methods for storage and delivery of such virus vectors. As described herein, the disclosed formulations provide compositions and methods for stable storage of AAV vectors (and other parvovirus vectors) at ambient temperatures. As disclosed herein, AAV vectors stabilized as disclosed herein retain significant biological activity even after extended storage conditions that would normally inactivate the compositions. Thus, the compositions of the disclosure offer significant advantages relative to previous formulations for AAV vectors, which may require refrigeration or even freezing to maintain activity for any significant period of time. This allows previously highly unstable compositions to be stored and transported over large distances with-out the typically required cold chain. Another advantage of the provided compositions is that the composition may foster storage of AAV vectors at concentrations that largely exceed solubility limits, but which may be desired for administration and/or use without compromising the physical stability and performance of the agent. This is a significant advantage over lyophilization and conventional formulations. Specific methods of generating the formulations described herein and also of using the formulations (e.g. formulations thereby generated), to deliver AAV vectors to a subject are also encompassed. Particular aspects are directed to injectable formulations and delivery of such formulations via intravenous methods.

### I. Compositions

In some aspects, the present disclosure provides a composition including, for example a virus, recombinant virus, viral vector, and/or components thereof, such as for use in gene delivery/gene therapy, vaccine delivery, etc., dispersed within a liquid or amorphous solid carrier, such as an amorphous film/thin film matrix. The virus, recombinant virus, viral vector, and/or components thereof, may include, for example, a whole organism, killed, attenuated or live (including killed, attenuated viruses); a subunit or portion of an organism; a recombinant vector containing an insert; a piece or fragment of a nucleic acid (DNA, RNA, etc.) associated with a virus, recombinant virus, viral vector, and/or components thereof; a protein, a polypeptide, a peptide associated with a virus, recombinant virus, viral vector, and/or components thereof, such as, for example, capsid proteins and/or empty capsids, virus particles and/or infectious particles, or any combination thereof. In some aspects, the virus may include any virus of the Parvoviridae family without departing from the scope of the disclosure. In some aspects of the compositions of the disclosure, the virus, recombinant virus, viral vector, and/or components thereof include adeno-associated virus (AAV), recombinant AAV, AAV vectors, and/or components thereof. Adeno-associated viruses within the scope of the disclosure include, for example, any natural AAV serotype (AAV1, AAV2, AAV3, AAV4, AAV5, AAV6, AAV7, AAV8, AAV9, etc.), as well as recombinant AAV including chimeric AAV capsids, AAV capsids with peptide insertions, capsids generated from AAV evolutionary libraries, or any AAV capsid used for gene delivery whether the AAV is derived from humans, primates, mammal, insects, etc., i.e., any species that supports AAV. In some aspects, the recombinant AAV may include capsids generated by artificial intelligence (AI). Exemplary non-parvoviruses that may be encompassed by the disclosure include, for example, Adenoviruses, Herpesviruses, Lentiviruses, Retroviruses, or any type of viral vector by which one of skill in the art would contemplate for gene delivery.

In some aspects, the virus, recombinant virus, viral vector, and/or components thereof may include a heterologous nucleic acid that encodes for a heterologous polypeptide that may have biological function and/or activity, and/or may include a heterologous nucleic acid that encodes for a heterologous nucleic acid that may have biological function and/or activity, for example, but not limited to, RNAi, crRNA, enhancer RNA, long non-coding RNA, microRNA, sRNA, and/or shRNA. For example, in some aspects, the heterologous nucleic acid may express a polypeptide having biological function and/or activity, and express a microRNA that directs/targets expression of the polypeptide to particular cells, tissues and/or organs in a subject.

In some aspects, between 1×10⁶ to about 1×10¹⁶ vg/ml are included in the formulation. In some aspects, 1×10¹⁵ vg/ml are included in the formulation. In some aspects, greater than 1×10⁶ vg/ml are included. In some aspects, greater than 1×10⁷ vg/ml are included. In some aspects, greater than 1×10⁸ vg/ml are included. In some aspects, greater than 1×10⁹ vg/ml are included. In some aspects, greater than 1×10¹⁰ vg/ml are included. In some aspects, greater than 1×10¹¹ vg/ml are included. In some aspects, greater than 1×10¹² vg/ml are included. In some aspects, greater than 1×10¹³ vg/ml are included. In some aspects, greater than 1×10¹⁴ vg/ml are included. In some aspects, greater than 1×10¹⁵ vg/ml are included. When the formulation is in non-liquid form (e.g., substantially solid film) the measurement of AAV vector may be referred to as the vg/ml of the liquid prior to conversion into the non-liquid form, for convenience. As discussed herein, when the amount of AAV vector is referred to in a composition in non-liquid form, in units of vg/ml, this is what is intended.

Without being bound by theory, it is believed that the disclosed formulations increase the ability to store higher concentrations of AAV vector while avoiding aggregation, and therefore allows storage and use at higher concentrations.

A composition of the disclosure may comprise 1, 2, 3, 4, or more different components disclosed herein. For example, a composition of the disclosure may comprise a viral vector and an additional component, such as a peptide or small molecule. A composition of the disclosure may comprise two or more components (e.g., a viral vector and a peptide, a viral vector and a small molecule, etc.) where all of the components in the composition retain biological activity (e.g., immunogenicity, biological activity, etc.) when stored at ambient temperature (e.g., between 15° C. and 30° C. for at least 1, 2, 3, 4, 5, 6, 7, 8, 9, 10, 20, 30, 40, 50, 60, 70, 80, 90, or 100 days, or more, or any range or value derivable therein.

In some aspects, a composition of the disclosure is an amorphous solid, such as a substantially solid film. In some aspects, an amorphous solid suitable for use in the present disclosure is dissolvable upon contact with an aqueous liquid. In some aspects, amorphous solids suitable for use in the present disclosure may be formed from any sugar, sugar derivative or combination of sugars/derivatives so long as the sugar and/or derivative is prepared as a liquid solution at a concentration that allows it to flow freely when poured but also forms an amorphous phase at ambient temperatures on a physical surface that facilitates this process, such as aluminum or Teflon. Examples of suitable sugars include, but are not limited to glucose, dextrose, fructose, lactose, maltose, xylose, sucrose, corn sugar syrup, sorbitol, hexitol, maltilol, xylitol, mannitol, melezitose, raffinose, and a combination thereof. In some aspects, a composition of the disclosure comprises sorbitol. In some aspects, a composition of the disclosure comprises a sugar that is not sorbitol. In certain aspects, it may be desirable for the properties of the sugar and/or derivative to allow preparation as a liquid solution at a concentration that allows it to flow freely when poured but also forms an amorphous phase at ambient temperatures on a physical surface that facilitates this process, such as aluminum or Teflon. While not being bound to any particular theory, it is believed that sugars minimize interaction of the virus, viral vector, or other molecules (e.g., polypeptide, nucleic acid, antigen, antibody, small molecule) with water during storage and drying, in turn, preventing damage to the three-dimensional shape due to crystal formation during the drying process and subsequent loss of efficacy. In some aspects, an amorphous solid suitable for use in the present disclosure may have a thickness of about 0.05 millimeters to about 5 millimeters (including any range or value derivable therein). In some aspects, the amorphous solid may include an amount of moisture following drying. For example, amorphous solids of the disclosure may have a moisture content of about 1-15% after drying (including any range or value derivable therein).

In addition, in some aspects, certain sugars may also function as a binder which may provide “substance” to pharmaceutical preparations that contain small quantities of very potent medications for ease of handling/administration. They may also hold components together or promote binding to surfaces (like the film backing) to ease drug delivery and handling. Lastly, they may also contribute to the overall pharmaceutical elegance of a preparation by forming uniform glasses upon drying.

Compositions of the present disclosure also may include a water-soluble polymer including, but not limited to, carboxymethyl cellulose, carboxyvinyl polymers, high amylose starch, hydroxyethyl cellulose, hydroxypropyl cellulose, hydroxypropyl methylcellulose (HPMC), methylmethacrylate copolymers, polyacrylic acid, polyvinyl alcohol, polyvinyl pyrrolidone, pullulan, sodium alginate, poly(lactic-co-glycolic acid), poly(ethylene) oxide, poly(hydroxyalkanoate) and a combination thereof.

In some aspects, the water-soluble polymer is chosen to provide particular characteristics to the composition. In some aspects, the water-soluble polymer is chosen to provide particular characteristics to the composition, for example, following reconstitution in solution. In some aspects, the water-soluble polymer is HPMC. Grades of HPMC encompassed by the disclosure include, for example, more viscous grades, such as K4M, E10M, and/or J75MS HPMC, as well as less viscous grades, such as K100LV, A4M, A15LV, E4M, F4M, E6LV (also “E6 Premium LV”), and/or F50 HPMC, but are not limited thereto.

It is envisioned that varying molecular weight HPMC may be used in the formulations and methods described herein. Varying the molecular weight of the HPMC in the composition will result in different viscosities of the formulation. In some aspects, the HPMC has a molecular weight that produces a viscosity of from about 12 cp to about 4000 cp (including any range or value derivable therein) at a concentration of 2% in water. In some aspects, the HPMC has a MW that produces a viscosity of less than 4000 cp, less than 3000 cp, less than 2000 cp, less than 1800 cp, or less than 400 cp at a concentration of 2% in water. In some aspects, the HPMC has a MW that produces a viscosity of from about 12 cp to about 3000 cp or from about 12 cp to about 1800 cp at a concentration of 2% in water. In some aspects, the HPMC has a MW that produces a viscosity of from about 12 cp to about 400 cp, from about 12 cp to about 100 cp, or from about 100 cp to about 400 cp at a concentration of 2% in water. In some aspects, a formulation, solution, or composition of the disclosure comprises an HPMC having a molecular weight that produces a viscosity that is at least, at most, about, or exactly 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000, 1010, 1020, 1030, 1040, 1050, 1060, 1070, 1080, 1090, 1100, 1110, 1120, 1130, 1140, 1150, 1160, 1170, 1180, 1190, 1200, 1210, 1220, 1230, 1240, 1250, 1260, 1270, 1280, 1290, 1300, 1310, 1320, 1330, 1340, 1350, 1360, 1370, 1380, 1390, 1400, 1410, 1420, 1430, 1440, 1450, 1460, 1470, 1480, 1490, 1500, 1510, 1520, 1530, 1540, 1550, 1560, 1570, 1580, 1590, 1600, 1610, 1620, 1630, 1640, 1650, 1660, 1670, 1680, 1690, 1700, 1710, 1720, 1730, 1740, 1750, 1760, 1770, 1780, 1790, 1800, 1810, 1820, 1830, 1840, 1850, 1860, 1870, 1880, 1890, 1900, 1910, 1920, 1930, 1940, 1950, 1960, 1970, 1980, 1990, 2000, 2010, 2020, 2030, 2040, 2050, 2060, 2070, 2080, 2090, 2100, 2110, 2120, 2130, 2140, 2150, 2160, 2170, 2180, 2190, 2200, 2210, 2220, 2230, 2240, 2250, 2260, 2270, 2280, 2290, 2300, 2310, 2320, 2330, 2340, 2350, 2360, 2370, 2380, 2390, 2400, 2410, 2420, 2430, 2440, 2450, 2460, 2470, 2480, 2490, 2500, 2510, 2520, 2530, 2540, 2550, 2560, 2570, 2580, 2590, 2600, 2610, 2620, 2630, 2640, 2650, 2660, 2670, 2680, 2690, 2700, 2710, 2720, 2730, 2740, 2750, 2760, 2770, 2780, 2790, 2800, 2810, 2820, 2830, 2840, 2850, 2860, 2870, 2880, 2890, 2900, 2910, 2920, 2930, 2940, 2950, 2960, 2970, 2980, 2990, 3000, 3010, 3020, 3030, 3040, 3050, 3060, 3070, 3080, 3090, 3100, 3110, 3120, 3130, 3140, 3150, 3160, 3170, 3180, 3190, 3200, 3210, 3220, 3230, 3240, 3250, 3260, 3270, 3280, 3290, 3300, 3310, 3320, 3330, 3340, 3350, 3360, 3370, 3380, 3390, 3400, 3410, 3420, 3430, 3440, 3450, 3460, 3470, 3480, 3490, 3500, 3510, 3520, 3530, 3540, 3550, 3560, 3570, 3580, 3590, 3600, 3610, 3620, 3630, 3640, 3650, 3660, 3670, 3680, 3690, 3700, 3710, 3720, 3730, 3740, 3750, 3760, 3770, 3780, 3790, 3800, 3810, 3820, 3830, 3840, 3850, 3860, 3870, 3880, 3890, 3900, 3910, 3920, 3930, 3940, 3950, 3960, 3970, 3980, 3990, 4000, 4010, 4020, 4030, 4040, 4050, 4060, 4070, 4080, 4090, 4100, 4110, 4120, 4130, 4140, 4150, 4160, 4170, 4180, 4190, 4200, 4210, 4220, 4230, 4240, 4250, 4260, 4270, 4280, 4290, 4300, 4310, 4320, 4330, 4340, 4350, 4360, 4370, 4380, 4390, 4400, 4410, 4420, 4430, 4440, 4450, 4460, 4470, 4480, 4490, 4500, 4510, 4520, 4530, 4540, 4550, 4560, 4570, 4580, 4590, 4600, 4610, 4620, 4630, 4640, 4650, 4660, 4670, 4680, 4690, 4700, 4710, 4720, 4730, 4740, 4750, 4760, 4770, 4780, 4790, 4800, 4810, 4820, 4830, 4840, 4850, 4860, 4870, 4880, 4890, 4900, 4910, 4920, 4930, 4940, 4950, 4960, 4970, 4980, 4990, or 5000 cp at a concentration of 2% in water, including any range or value derivable therein.

Examples of HPMC of varying molecular weights are known and available in the art, and include, without limitation, A4M, F4M, A15C, A4C, K100LV, A15LV, and E6LV (also “E6 Premium LV”). HPMC of a single molecular weight may be used, or the use of a combination of various molecular weight HPMC, e.g. as disclosed herein, is also envisioned. A composition of the disclosure may comprise at least, at most, about, or exactly 0.1, 0.2, 0.3, 0.4, 0.5, 0.6, 0.7, 0.8, 0.9, 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3, 3.1, 3.2, 3.3, 3.4, 3.5, 3.6, 3.7, 3.8, 3.9, 4, 4.1, 4.2, 4.3, 4.4, 4.5, 4.6, 4.7, 4.8, 4.9, or 5% HPMC, including any range or value derivable therein. In some aspects, a composition of the disclosure comprises 1.0%, 1.5%, 2.0% HPMC, or any range or value derivable therein. In some aspects, a composition of the disclosure comprises about or exactly 1.5% HPMC.

Other water soluble polymers may be substituted in part or in whole for HPMC. Examples of suitable water soluble polymers include, without limitation, carboxymethyl cellulose, carboxyvinyl polymers, high amylase starch, hydroxyethyl cellulose, hydroxypropyl cellulose, hydroxypropyl methylcellulose, methylmethacrylate copolymers, polyacrylic acid, polyvinyl alcohol, polyvinyl pyrrolidone, pullulan, sodium alginate, poly(lactic-co-glycolic acid), poly(ethylene) oxide, poly(hydroxyalkanoate), and combinations thereof.

Furthermore, in some aspects, the compositions of the present disclosure may further include one or more oils, polyalcohols, surfactants, permeability enhancers, and/or edible organic acids. Examples of suitable oils may include, but are not limited to, eucalyptol, menthol, vacrol, thymol, methyl salicylate, verbenone, eugenol, gerianol and a combination thereof. Examples of suitable polyalcohols may include, but are not limited to, glycerol, polyethylene glycol, propylene glycol, and a combination thereof. In some aspects, a composition of the disclosure comprises glycerol. In some aspects, a composition of the disclosure comprises a polyalcohol that is not glycerol. Examples of suitable edible organic acids may include, but are not limited to, citric acid, malic acid, tartaric acid, fumaric acid, phosphoric acid, oxalic acid, ascorbic acid and a combination thereof. Examples of suitable surfactants may include, but are not limited to, difunctional block copolymer surfactants terminating in primary hydroxyl groups, such as Pluronic® F68 commercially available from BASF, poly(ethylene) glycol 3000, dodecyl-ß-D-maltopyranoside, disodium PEG-4 cocamido MIPA-sulfosuccinate (DMPS), etc.

In some aspects, a composition of the disclosure may include a zwitterionic molecule, such as a zwitterionic surfactant, or an amino acid or an amino acid derivative. In some aspects, the zwitterionic surfactant is a surfactant molecule which contains a group which is capable of being positively charged and a group which is capable of being negatively charged. In some aspects, both the positively charged and negatively charged groups are ionized at physiological pH such that the molecule has a net neutral charge. In some aspects, the positively charged group includes a protonated or quaternary ammonium. In some aspects, the negatively charged group includes a sulfate, a phosphate, or a carboxylate. The zwitterionic surfactant further includes one or more lipid groups consisting essentially of an alkyl, cycloalkyl, or alkenyl groups. Preferably, the zwitterionic surfactant includes one or more lipid groups consisting essentially of an alkyl, cycloalkyl, or alkenyl groups with a carbon chain of more than 12 carbon atoms. In some aspects, the lipid group has a carbon chain of 12-30 carbon atoms. In some aspects, the lipid group has a carbon chain of 12-24 carbon atoms. In some aspects, the lipid group has from 12, 13, 14, 15, 16, 17, 18, 19, 20, 21, 22, 23, to 24 carbons, or any range derivable thereof. In some aspects, the zwitterionic surfactant is a polymeric structure which contains multiple zwitterionic groups and multiple lipid groups on a central backbone. In some aspects, the zwitterionic surfactant is a polymer which has from about 50 to about 200 repeating units wherein each repeating unit includes one positively charged group, one negatively charged group, and one lipid group. In some aspects, the zwitterionic surfactant is a polymer which has a 75 to 150 repeating units. In some aspects, the central backbone is an alkyl, polyethylene glycol, or polypropylene chain. In some aspects, the central chain is an alkyl group.

Non-limiting examples of zwitterionic surfactants include 3-(N,N-Dimethyltetradecylammonio)propanesulfonate (SB3-14), 3-(4-Heptyl)phenyl-3-hydroxypropyl)dimethylammoniopropanesulfonate (C7BzO), 3-(decyldimethylammonio) propanesulfonate inner salt (SB3-10), 3-(dodecyldimethylammonio) propanesulfonate inner salt (SB3-12), 3-(N,N-dimethyloctadecylammonio) propanesulfonate (SB3-18), 3-(N,N-dimethyl-octylammonio) propanesulfonate inner salt (SB3-8), 3-(N,N-dimethylpalmitylammonio) propanesulfonate (SB3-16), 3-[N,N-dimethyl(3-myristoylaminopropyl)ammonio]propane-sulfonate (ASB-14), CHAPS, CHAPSO, acetylated lecithin, alkyl(C12-30) dialkylamine-N-oxide apricotamidopropyl betaine, babassuamidopropyl betaine, behenyl betaine, bis 2-hydroxyethyl tallow glycinate, C12-14 alkyl dimethyl betaine, canolamidopropyl betaine, capric/caprylic amidopropyl betaine, capryloamidopropyl betaine, cetyl betaine, 3-[(Cocamidoethyl)dimethylammonio]-2-hydroxypropanesulfonate, 3-[(Cocamidoethyl)dimethyl-ammonio]propanesulfonate, cocamidopropyl betaine, cocamidopropyl dimethylamino-hydroxypropyl hydrolyzed collagen, N-[3-cocamido)-propyl]-N,N-dimethyl betaine, potassium salt, cocamidopropyl hydroxysultaine, cocamidopropyl sulfobetaine, cocaminobutyric acid, cocaminopropionic acid, cocoamphodipropionic acid, coco-betaine, cocodimethylammonium-3-sulfopropylbetaine, cocoiminodiglycinate, cocoiminodipropionate, coco/oleamidopropyl betaine, cocoyl sarcosinamide DEA, DEA-cocoamphodipropionate, dihydroxyethyl tallow glycinate, dimethicone propyl PG-betaine, N,N-dimethyl-N-lauric acid-amidopropyl-N-(3-sulfopropyl)-ammonium betaine, N,N-dimethyl-N-myristyl-N-(3-sulfopropyl)-ammonium betaine, N,N-dimethyl-N-palmityl-N-(3-sulfopropyl)-ammonium betaine, N,N-dimethyl-N-stearamidopropyl-N-(3-sulfopropyl)-ammonium betaine, N,N-dimethyl-N-stearyl-N-(3-sulfopropyl)-ammonium betaine, N,N-dimethyl-N-tallow-N-(3-sulfopropyl)-ammonium betaine, disodium caproamphodiacetate, disodium caproamphodipropionate, disodium capryloamphodiacetate, disodium capryloamphodipropionate, disodium cocoamphodiacetate, disodium cocoamphodipropionate, disodium isostearoamphodipropionate, disodium laureth-5 carboxyamphodiacetate, disodium lauriminodipropionate, disodium lauroamphodiacetate, disodium lauroamphodipropionate, disodium octyl b-iminodipropionate, disodium oleoamphodiacetate, disodium oleoamphodipropionate, disodium PPG-2-isodeceth-7 carboxyamphodiacetate, disodium soyamphodiacetate, disodium stearoamphodiacetate, disodium tallamphodipropionate, disodium tallowamphodiacetate, disodium tallowiminodipropionate, disodium wheatgermamphodiacetate, N,N-distearyl-N-methyl-N-(3-sulfopropyl)-ammonium betaine, erucamidopropyl hydroxysultaine, ethylhexyl dipropionate, ethyl hydroxymethyl oleyl oxazoline, ethyl PEG-15 cocamine sulfate, hydrogenated lecithin, hydrolyzed protein, isostearamidopropyl betaine, 3-[(Lauramidoethyl)dimethylammonio]-2-hydroxypropane-sulfonate, 3-[(Lauramidoethyl)dimethylammonio]propanesulfonate, lauramido-propyl betaine, lauramidopropyl dimethyl betaine, lauraminopropionic acid, lauroamphodipropionic acid, lauroyl lysine, lauryl betaine, lauryl hydroxysultaine, lauryl sultaine, linoleamidopropyl betaine, lysolecithin, milk lipid amidopropyl betaine, myristamidopropyl betaine, octyl dipropionate, octyliminodipropionate, n-octyl-N,N-dimethyl-3-ammonio-1-propanesulfonate, n-decyl-N,N-dimethyl-3-ammonio-1-propanesulfonate, n-dodecyl-N,N-dimethyl-3-ammonio-1-propane-sulfonate, n-tetradecyl-N,N-dimethyl-3-ammonio-1-propanesulfonate, n-hexadecyl-N,N-dimethyl-3-ammonio-1-propanesulfonate, n-octadecyl-N,N-dimethyl-3-ammonio-1-propane-sulfonate, oleamidopropyl betaine, oleyl betaine, 4,4(5H)-oxazoledimethanol, 2-(heptadecenyl) betaine, palmitamidopropyl betaine, palmitamine oxide, PMAL-C6, PMAL-C12, PMAL-C16, ricinoleamidopropyl betaine, ricinoleamidopropyl betaine/IPDI copolymer, sesamidopropyl betaine, sodium C12-15 alkoxypropyl iminodipropionate, sodium caproamphoacetate, sodium capryloamphoacetate, sodium capryloamphohydroxypropyl sulfonate, sodium capryloamphopropionate, sodium carboxymethyl tallow polypropylamine, sodium cocaminopropionate, sodium cocoamphoacetate, sodium cocoamphohydroxypropyl sulfonate, sodium cocoamphopropionate, sodium dicarboxyethyl cocophosphoethyl imidazoline, sodium hydrogenated tallow dimethyl glycinate, sodium isostearoamphopropionate, sodium lauriminodipropionate, sodium lauroamphoacetate, sodium oleoamphohydroxypropylsulfonate, sodium oleoamphopropionate, sodium stearoamphoacetate, sodium tallamphopropionate, soyamidopropyl betaine, stearyl betaine, 3-[(Stearamidoethyl)dimethylammonio]-2-hydroxypropanesulfonate, 3-[(Stearamidoethyl)-dimethylammoniolpropanesulfonate, tallowamidopropyl hydroxysultaine, tallowamphopoly-carboxypropionic acid, trisodium lauroampho PG-acetate phosphate chloride, undecylenamidopropyl betaine, and wheat germamidopropyl betaine. In some aspects, the zwitterionic surfactant is PMAL-C16.

In some aspects, the zwitterionic molecule may be an amino acid and/or an amino acid derivative, such as any natural or artificial/synthetic amino acid and/or amino acid derivative, including but not limited to Alanine, Arginine, Asparagine, Aspartic Acid, Glutamic Acid, Cysteine, Glutamine, Glycine, Histidine, Isoleucine, Leucine, Lysine, Methionine, Phenylalanine, Proline, Selenocysteine, Serine, Threonine, Tryptophan, Tyrosine, Valine, Citrulline, Ornithine, Theanine, Betaine, Carnitine, Taurine, Tyramine, and/or Gamma Aminobutyric Acid, and/or any derivative thereof without limitation.

Components of the compositions other than the virus, recombinant virus, viral vector, and/or components thereof as described herein (e.g., AAV vector) are exemplified in, for example, PCT International Publication No. WO 2012/018628 and U.S. Patent Application Publication No. 2019/0298836, incorporated by reference herein.

In some aspects of the invention, the formulation is a pharmaceutical composition comprising between 1×10⁶ to about 1×10¹⁶ vg/ml of an AAV vector formulated within: from about 0.1% to about 5% wt/vol HPMC (including any range or value derivable therein); from about 0.5% to about 5% glycerol (including any range or value derivable therein); from about 0.5% to about 5% sorbitol (including any range or value derivable therein); and from about 0.1% to about 5% PMAL-C16 (including any range or value derivable therein); wherein, the HPMC has a MW that produces a viscosity that is less than 4000 cp at a concentration of 2% in water, and, wherein the pH is adjusted so that the final pH of the composition is from about pH 6.0 to 8.5 (including any range or value derivable therein).

Adjustment to a desired pH may be accomplished with a suitable buffer such as phosphate buffered saline (PBS) and tris (Tris(hydroxymethyl)aminomethane) buffer. In some aspects, the buffer or combination thereof, is added so that the final pH is about 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, or 8.5 (including any range or value derivable therein. In some aspects, a composition of the present disclosure has a pH of at least, at most, or about 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, or 8.5. In some aspects, the pH of a composition of the disclosure is at least 7.0. In some aspects, the pH is between 7.5 and 8.5. In certain aspects, the pH is about or exactly 8.1.

In one particular aspect, a composition of the disclosure is a composition comprising a virus, recombinant virus, viral vector, and/or components thereof (e.g., an AAV vector), further comprising about or exactly 1.5% K4M HPMC, about or exactly 2% sorbitol, about or exactly 2% glycerol, about or exactly 1% PMAL-C16, and about or exactly 10 mM Tris and having a pH of about or exactly 8.1.

In one particular aspect, a composition of the disclosure is a composition comprising a virus, recombinant virus, viral vector, and/or components thereof (e.g., an AAV vector), further comprising about or exactly 1.5% A4M HPMC, about or exactly 2% sorbitol, about or exactly 2% glycerol, about or exactly 1% PMAL-C16, and about or exactly 10 mM Tris and having a pH of about or exactly 8.1.

In one particular aspect, a composition of the disclosure is a composition comprising a virus, recombinant virus, viral vector, and/or components thereof (e.g., an AAV vector), further comprising about or exactly 1.5% F4M HPMC, about or exactly 2% sorbitol, about or exactly 2% glycerol, about or exactly 1% PMAL-C16, and about or exactly 10 mM Tris and having a pH of about or exactly 8.1.

In one particular aspect, a composition of the disclosure is a composition comprising a virus, recombinant virus, viral vector, and/or components thereof (e.g., an AAV vector), further comprising about or exactly 1.5% A15C HPMC, about or exactly 2% sorbitol, about or exactly 2% glycerol, about or exactly 1% PMAL-C16, and about or exactly 10 mM Tris and having a pH of about or exactly 8.1.

In one particular aspect, a composition of the disclosure is a composition comprising a virus, recombinant virus, viral vector, and/or components thereof (e.g., an AAV vector), further comprising about or exactly 1.5% A4C HPMC, about or exactly 2% sorbitol, about or exactly 2% glycerol, about or exactly 1% PMAL-C16, and about or exactly 10 mM Tris and having a pH of about or exactly 8.1.

In one particular aspect, a composition of the disclosure is a composition comprising a virus, recombinant virus, viral vector, and/or components thereof (e.g., an AAV vector), further comprising about or exactly 1.5% A15LV HPMC, about or exactly 2% sorbitol, about or exactly 2% glycerol, about or exactly 1% PMAL-C16, and about or exactly 10 mM Tris and having a pH of about or exactly 8.1.

In one particular aspect, a composition of the disclosure is a composition comprising a virus, recombinant virus, viral vector, and/or components thereof (e.g., an AAV vector), further comprising about or exactly 1.5% E6LV (also “E6 Premium LV”) HPMC, about or exactly 2% sorbitol, about or exactly 2% glycerol, about or exactly 1% PMAL-C16, and about or exactly 10 mM Tris and having a pH of about or exactly 8.1.

In one particular aspect, a composition of the disclosure is a composition comprising a virus, recombinant virus, viral vector, and/or components thereof (e.g., an AAV vector), further comprising about or exactly 1.5% HPMC that is not K4M HPMC, about or exactly 2% sorbitol, about or exactly 2% glycerol, about or exactly 1% PMAL-C16, and about or exactly 10 mM Tris and having a pH of about or exactly 8.1.

As described herein, the disclosed formulations, in both liquid and solid form, promote significant preservation of the AAV vector contained therein to thereby stabilize the AAV vector for extended periods of time. This results in the AAV vector composition retaining significantly more activity than with other forms of storage. For example, stabilization for lengths of time of at least 3 years when stored at room temperature, are expected. These formulations, or forms of the formulation (e.g., liquid or solid form) promote significant preservation of the AAV vector when stored for 1 or more weeks, 2 or more weeks, 3 or more weeks, 4 or more weeks, 5 or more weeks. Storage at temperatures at or above 0° C. are envisioned (e.g., at least, about, or exactly 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40° C., including any range or value derivable therein).

A significant amount of preservation is considered to be that which results in a usable amount of viral vector for the intended purpose (e.g., gene therapy to deliver nucleic acid, or diagnostic purposes). It is believed that considerable value is obtained even with a composition described herein that results in 45% or less (e.g., about 40%, about 35%, about 30%, about 25%, etc.) preservation of AAV vector activity, in light of the ease of storage and shipment.

The amount of preserved viral vector can be determined by comparison of active viral vector in the stored form versus the original amount of active viral vector, percent (%) recovery of infectious virus after storage. Activity can be measured by various methods known in the art such as virus infectivity, e.g., as measured by viral transduction efficiency and/or viral vector genome copy number, and/or percent recovery of infectious virus following storage. In some aspects, viral vector activity is preserved by at least 20%, 25%, 30%, 35%, 40%, 45%, 50%, 55%, 60%, 65%, 70%, 75%, 80%, 85%, 90%, 95%, or 99%. In some aspects, AAV infectivity is preserved by at least 50%, at least 60%, at least 70%, at least 80%, at least 90% or, more, after exposure to conditions up to 95% relative humidity and up to 40° C. temperature. In some aspects, AAV infectivity is preserved by at least 50%, at least 60%, at least 70%, at least 80%, at least 90% or, more, for 150 days or, more.

One method of determining the preservation is by comparing the activity (e.g., as measured by genome copy number, infectivity, transduction efficiency, etc.) of the AAV vector prior to storage (e.g., prior to or just after adding to the rest of the composition components, or just after film formation) to the activity after storage.

Various conditions may be encountered during the storage, such as storage at about 4ºC, storage at about 0° C., storage at about 25° C., combinations of different temperatures, exposure to extreme temperatures, freeze thawing, or combinations thereof. In some aspects, storage it at ambient temperature. In some aspects varying temperatures are encountered during storage, ranging from 4° C. to 40° C. or higher. Such variations may be encountered during shipping, and/or storage in areas that lack refrigeration. In some aspects, the stored formulations are subjected to ambient temperature (e.g., e.g., 20° C. +/−10°, +/−5°, +/−4°, +/−3°, +/−2°, +/−1° C.) and ambient pressure (e.g., approximately 1 atm, +/−0.5 atm). The formulations and methods described herein are expected to provide significant preservation of AAV vector under varying conditions, including different relative humidity (e.g. 5% RH to 99% RH, such as about 20%, about 30%, about 40%, about 50%, about 60%, about 70%, about 80%, about 90% RH, about 95% RH), and different pressures.

### II. Methods of Preparation and Use

In some aspects, a composition including an amorphous solid may be made by preparing a solution including a sugar, sugar derivative or combination of sugars/derivatives in a buffer and optionally other additives previously mentioned. In some aspects, a sugar, sugar derivative or combination of sugars/derivatives may be present in the solution in an amount up to about 50%, about 60%, about 70%, or up to about 80% by weight of the solution. In some aspects, an additive may be present in an amount of about 5% or less, about 4% or less, about 3% or less, about 2% or less, or 1% or less by weight of the solution. In general, the solution including the sugar, sugar derivative or combination of sugars/derivatives is made at a concentration higher than the desired final concentration to compensate for any dilution that may occur when the virus, viral vector, or other molecules (e.g., peptide, antigen) is added. The desired virus, viral vector, or other molecule (e.g., peptide, antigen) may be added to the solution at a concentration known to induce the desired immune response. In some aspects, the solution has a pH of at least, at most, or about 5, 6, 7, 8, 9, or 10, or any range or value derivable therein. In some aspects, the solution has a pH between 7 and 9, including any range or value derivable therein. In some aspects, the solution has a pH of about or exactly 7, 7.1, 7.2, 7.3, 7.4, 7.5, 7.6, 7.7, 7.8, 7.9, 8, 8.1, 8.2, 8.3, 8.4, 8.5, 8.6, 8.7, 8.8, 8.9, or 9, or any value derivable therein. In some aspects, the solution has a pH of about 8. In some aspects, the solution has a pH of 8. In some aspects, the solution has a pH of about 8.1. In some aspects, the solution has a pH of 8.1.

The mixture may then be stirred at ambient temperature until a substantially homogeneous mixture is obtained. In some aspects, the mixture may then be briefly sonicated under cooled conditions, e.g. 4° C., to remove any air bubbles that may have developed. In other embodiments, the mixture may be slightly heated, e.g., heated to about 40° C. or below, about 45° C. or below, or about 50° C. or below, slightly cooled, and in some instances may be frozen. In some aspects, a composition of the present disclosure (e.g., AAV-containing composition) may be made without free drying or spray draying. The final formulation may then be cast onto a flat backing surface under controlled air flow, such as a controlled, laminar flow of air and allowed to form an amorphous solid at ambient temperatures (e.g., about 15-25° C.). Examples of suitable backing surfaces may include, but are not limited to, aluminum, Teflon, silicate, polyetheretherketone, polyethylene, polypropylene, polyvinylchloride, polyamide, polyacrylate, polyester, ethyl cellulose, and silicone, including any combination thereof. Once the process is complete the composition can be peeled from the backing and administered immediately (e.g., by placement in the mouth or by injection) and/or stored at ambient temperature for up to about 7 days, up to about 14 days, up to about one month (about 30 days), up to about two months (about 60 days), up to about three months (about 90 days), up to about four months (about 120 days), up to about five months (about 150 days), up to about six months (about 180 days), up to about one year (about 50 weeks), up to about two years (about 100 weeks), or even up to about three years (about 150 weeks) from manufacture.

In some aspects, the composition of the present disclosure may be made by contacting an amorphous solid with a virus, recombinant virus, viral vector, and/or components thereof (e.g., an AAV vector), or optionally, mixing a virus, recombinant virus, viral vector, and/or components thereof with one or more excipients (surfactants, sugars, starches, etc.) and contacting the amorphous solid with the mixture so as to dispose the virus, recombinant virus, viral vector, and/or components thereof within the amorphous solid. In some aspects, the mixture is then allowed to dry, which is then ready for administration. A composition of the disclosure can be used in a variety of ways (e.g., as a therapeutic delivery vehicle such as for gene therapy, as a vaccine capable of eliciting an immune response from the immune system of a subject receiving the composition, for storage of one or more biological components such as nucleic acids or peptides, etc.).

In some aspects, compositions of the present disclosure may further include a protective layer disposed on a surface of an amorphous solid including a virus, recombinant virus, viral vector, and/or components thereof (e.g., an AAV vector). Exemplary protective layers may include, but are not limited to, an additional layer(s) of film, such as polyethylene, polyurethane, polyether etherketone, etc., and/or an additional layer(s) of an amorphous solid that does not contain any virus, recombinant virus, viral vector, and/or components thereof. In some aspects, the use of a protective layer of film may minimize the absorption of moisture from the atmosphere and prevent adherence to other objects during storage and/or transport. Prior to administration, this layer may be removed from the device (e.g., by peeling the layer off) and may be discarded.

The amount of virus, recombinant virus, viral vector, and/or components thereof that may be used in a composition of the present disclosure may vary greatly depending upon the type of virus, recombinant virus, viral vector, and/or components thereof being used, the formulation being used to prepare the composition, the size of the amorphous solid, solubility, etc. One of ordinary skill in the art with the benefit of this disclosure will be able to determine a suitable amount of virus, recombinant virus, viral vector, and/or components thereof to include in a composition of the present disclosure. In embodiments of the disclosure, compositions may include, for example, about 1×10⁶ to about 1×10¹³ virus particles.

It is also important to note that when formulating a composition according to the present disclosure, one must also consider any toxicity and/or adverse effects. Furthermore, in an effort to create a stable composition, it may also be important to identify a ratio of ingredients that interacts with water and the molecule (e.g., AAV vector) in a manner that prevents crystallization during drying.

In some aspects, a glass plate can be used for casting of the composition, which can be dried under a controlled, laminar flow of air at room temperature, or under refrigerated conditions. Similarly, compositions suitable for use according to the present disclosure can be prepared in a single-layer or multi-layers.

In general, the compositions of the present disclosure may be formulated so as to dissolve from about 5 to 60 seconds up to a period of 2 hours. When administered, a composition of the present disclosure may be handled by a portion of the composition that does not contain the virus, recombinant virus, viral vector, and/or components thereof, and may be placed in the upper pouch of the cheek for buccal delivery, or far under the tongue for sublingual delivery, or reconstituted and utilized, for example as a solution for inhalation or as a nasal spray.

Reconstitution of the compositions of the disclosure, such as a thin film matrix, may be accomplished, for example, by solubilizing in, for example, saline; PBS; salt solutions; formulations that require specific sugar or lipid based excipients; traditional solutions used in IV reconstitution of medicinal agents, body fluids; or any other fluid matrix that allows the compositions of the disclosure to reconstitute the virus, recombinant virus, viral vector, and/or components thereof (e.g., AAV vector) preserved in such a matrix.

In some aspects, methods are provided for producing compositions in substantially solid carriers. Such a method may include obtaining or formulating a solution including sufficient stabilizers (e.g., sugars and sugar derivatives, polymers) and permeability enhancers (e.g., surfactants, such as a zwitterionic surfactant of the embodiments) in a solvent system (e.g., distilled deionized water, ethanol, methanol). In some cases, formulation is such that the total amount of solid components added to the solvent are within the concentration of 10%-90% w/w. This suspension can be prepared by stirring, homogenization, mixing and/or blending these compounds with the solvent. In some cases, small portions of each component (e.g., about 1/10 the total amount) are added to the solvent and the solution mixed before adding additional portions of the same agent or a new agent.

In some aspects, once each stabilizer and permeability enhancer is added, the bulk solution is placed at 4° C. for a period of time between 2-24 hours. In some aspects, the bulk solution is subjected additional homogenization, such as sonication (e.g., for a period of about 5-60 minutes) to remove trapped air bubbles in the preparation. After sonication is complete, the virus, recombinant virus, viral vector, end/or components thereof, for example a virus and/or infectious particle, is added to the preparation. In some cases, the amount of the virus and/or infectious particle will range from of about 0.1-30% of the total solid concentration.

In some aspects, the preparation is then slowly piped into molds of a shape suitable for the application. The molds can be constructed of a variety of materials including, but not limited to, stainless steel, glass, silicone, polystyrene, polypropylene and other pharmaceutical grade plastics. In some aspects, the preparation can be placed in the molds by slowly pouring by hand or by pushing the preparation through a narrow opening on a collective container at a slow controlled rate (e.g., about 0.25 ml/min) to prevent early hardening and/or bubble formation in the final film product. In certain preferred aspects, films can be poured to a thickness of about 12.5-1000 μm. In some aspects, molds for casting of films can be sterilized by autoclaving and placed in laminar air flow hoods prior to casting.

In further embodiments, molds may also be lined with a peelable backing material suitable for protection of the film product. Suitable backings include, without limitation, aluminum, gelatin, polyesters, polyethylene, polyvinyl and poly lactic co-glycolide polymers, wax paper and/or any other pharmaceutically acceptable plastic polymer.

In some cases, cast films will remain at ambient temperature (e.g., about 15-25° C.), such as in a laminar flow hood for 2-24 hours after which time a thin, peelable film will be formed. In some cases, this film may be opaque or translucent. In some cases, individual films are peeled from the casting/drying surface, wrapped in wax paper and stored at room temperature (e.g., between 15° C. and 30° C., between 18° C. and 28° C., between 24° C. and 26° C., or about 25° C.) in sealable plastic bags under controlled humidity conditions. However, in certain aspects, films can be stored at lower temperatures, such as at 4° C., under controlled humidity as well.

In some aspects, multilayer films can also be created at this time by applying a second coating of as solution containing the same virus, recombinant virus, viral vector, and/or components thereof, as the first layer or another different virus, recombinant virus, viral vector, and/or components thereof to the thin film. Again, in some aspects, this will remain at ambient temperature (e.g., about 15-30° C., about 20-25° C., about 18-28° C., or about 25° C.), such as in a laminar flow hood, for an additional 2-24 hours after which time a thin, peelable film will be formed. Again, the film may be opaque or translucent.

In some aspects, films will be dissolved in a solution prior to use. For example, water or warmed saline (e.g., about 37° C., body temperature) may be used. In some aspects, the resulting solution can be screened for activity/particle count to determine the effectiveness of the formulation to retain the potency of the preparation over time. Such a dissolved film may be administered, for example, via intravenous administration to a subject.

An AAV vector contained within the disclosed formulations can be further diluted as needed prior to administration (e.g., in a pharmaceutically acceptable carrier). The appropriate route of administration can be determined by the skilled practitioner.

As used herein, “pharmaceutically acceptable carrier” includes any and all solvents, dispersion media, coatings, surfactants, antioxidants, preservatives (e.g., antibacterial agents, antifungal agents), isotonic agents, absorption delaying agents, salts, preservatives, drugs, drug stabilizers, gels, binders, excipients, disintegration agents, lubricants, sweetening agents, flavoring agents, dyes, and like materials and combinations thereof, as would be known to one of ordinary skill in the art (see, for example, Remington's Pharmaceutical Sciences, 18th Ed., Mack Printing Company, 1990, pp. 1289-1329, incorporated herein by reference). Except insofar as any conventional carrier is incompatible with the active ingredient, its use in the therapeutic or pharmaceutical compositions is contemplated.

In some aspects, the herein described formulations are used to deliver an effective amount of AAV vector to a subject. As such, another aspect of the invention relates to a method of delivering an AAV vector to a subject comprising administering to the subject an effective amount of the composition described herein. In some aspects, the composition is administered directly. In some aspects, the composition is manipulated minimally (e.g., films are rehydrated then administered). In some aspects, the composition is even further diluted (e.g., with a pharmaceutically acceptable carrier).

In some aspects, such an effective amount is a therapeutically effective amount. A “therapeutically effective” amount as used herein is an amount that is sufficient to provide some improvement or benefit to the subject. Alternatively stated, a “therapeutically effective” amount is an amount that will provide some alleviation, mitigation, or decrease in at least one clinical symptom in the subject. Those skilled in the art will appreciate that the therapeutic effects need not be complete or curative, as long as some benefit is provided to the subject. In certain embodiments, the therapeutically effective amount is not curative.

The pharmaceutical composition of the invention may exploit different types of carriers depending on whether it is to be administered in solid, liquid or aerosol form, and whether it need be sterile for such routes of administration as injection. The pharmaceutical compositions can be administered intravenously, intradermally, intra-arterially, intra-graft, intraperitoneally, intralesionally, intracranially, intraspinally, intracistemally, intraarticularly, intraprostatically, intrapl eurally, intratracheally, intranasally, intravitreally, intravaginally, intrarectally, topically, intratumorally, intramuscularly, intraperitoneally, subcutaneously, subconjunctivally, intravesicularly, mucosally, intrapericardially, intraumbilically, intraocularly, orally, topically, locally, inhalation (e.g. aerosol inhalation), injection, infusion, continuous infusion, localized perfusion bathing target cells directly (e.g., in an autogenous tissue graft), via a catheter, via lavage, in cremes, in lipid compositions (e.g., liposomes), or by any other method or any combination of the foregoing as would be known to one of ordinary skill in the art (see, for example, Remington's Pharmaceutical Sciences, 18th Ed., Mack Printing Company, 1990). Administration into istema magna, ventricles in brain, intraparenchymal in brain, lumbar puncture, intrathecal, into ureter, into renal artery, subretinal, subpial, intracoronary (into the heart), direct injections into salivary gland, into sensory organ of the ears (e.g. to treat deafness), into the ganglia, intra-articular, into placenta/vein, and limb perfusions is also envisioned.

### III. General Pharmaceutical Compositions

The phrases “pharmaceutically acceptable” or “pharmacologically acceptable” refer to molecular entities and compositions that do not produce an adverse, allergic, or other untoward reaction when administered to an animal or human. As used herein, “pharmaceutically acceptable carrier” includes any and all solvents, dispersion media, coatings, anti-bacterial and anti-fungal agents, isotonic and absorption delaying agents, and the like. The use of such media and agents for pharmaceutical active substances is well known in the art. Except insofar as any conventional media or agent is incompatible with the active ingredients, its use in immunogenic and therapeutic compositions is contemplated. Supplementary active ingredients, such as other anti-infective agents and vaccines, can also be incorporated into the compositions.

The active compounds can be formulated for parenteral administration, e.g., formulated for injection via the intravenous, intramuscular, subcutaneous, or intraperitoneal routes. Typically, such compositions can be prepared as either liquid solutions or suspensions; solid forms suitable for use to prepare solutions or suspensions upon the addition of a liquid prior to injection can also be prepared; and, the preparations can also be emulsified. Also contemplated are substantially solid film formulations, as disclosed herein.

The pharmaceutical forms suitable for injectable use include sterile aqueous solutions or dispersions; formulations including, for example, aqueous propylene glycol; and sterile powders for the extemporaneous preparation of sterile injectable solutions or dispersions. In all cases the form must be sterile and must be fluid to the extent that it may be easily injected. It also should be stable under the conditions of manufacture and storage and must be preserved against the contaminating action of microorganisms, such as bacteria and fungi.

A pharmaceutical composition can include a solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion, and by the use of surfactants. The prevention of the action of microorganisms can be brought about by various anti-bacterial and anti-fungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions are prepared by incorporating the active compounds in the required amount in the appropriate solvent with various other ingredients enumerated above, as required, followed by filtered sterilization or an equivalent procedure. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, certain methods of preparation are vacuum-drying and freeze-drying techniques, which yield a powder of the active ingredient, plus any additional desired ingredient from a previously sterile-filtered solution thereof.

Administration of the disclosed compositions will typically be via any common route. This includes, but is not limited to oral, or intravenous administration. In some aspects, the disclosed compositions are particularly useful for intravenous administration, e.g., due to low viscosity (e.g., less than 4000, 3000, 2000, 1000, 500, or 250 cp, or less) Alternatively, administration may be by orthotopic, intradermal, subcutaneous, intramuscular, intraperitoneal, or intranasal administration. Such compositions would normally be administered as pharmaceutically acceptable compositions that include physiologically acceptable carriers, buffers or other excipients.

Upon formulation, solutions will be administered in a manner compatible with the dosage formulation and in such amount as is therapeutically or prophylactically effective. The formulations are easily administered in a variety of dosage forms, such as the type of injectable solutions described above.

### EXAMPLES

The following examples are included to demonstrate certain embodiments of the invention. It should be appreciated by those of skill in the art that the techniques disclosed in the examples which follow represent techniques discovered by the inventor to function well in the practice of the invention, and thus can be considered to constitute certain modes for its practice. However, those of skill in the art should, in light of the present disclosure, appreciate that many changes can be made in the specific embodiments which are disclosed and still obtain a like or similar result without departing from the spirit and scope of the invention.

### Example 1—Assessment of Short-Term Stability of Recombinant AAV in Current Formulation

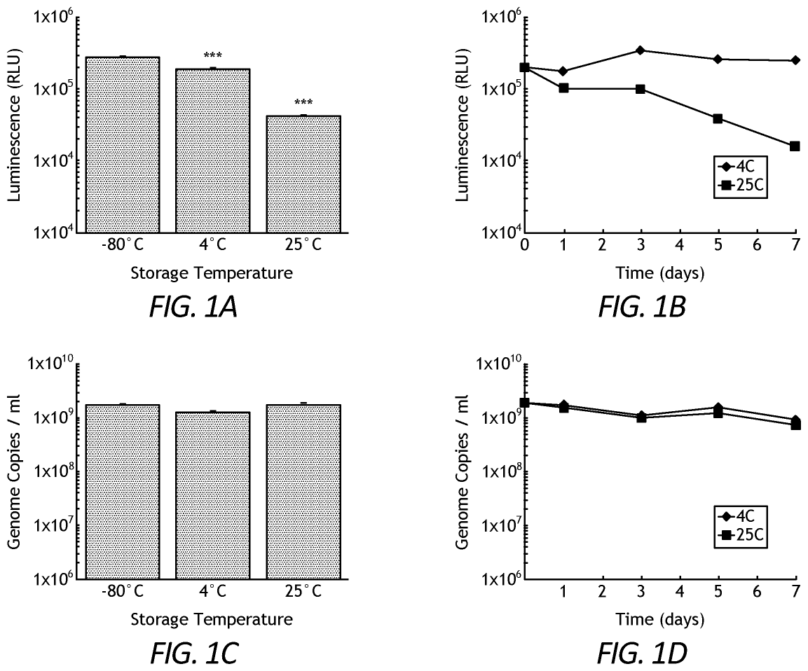

Prior to studying the stability of recombinant AAV9 within a film matrix, a small scale stability study was initiated to evaluate changes in vector potency when the currently formulated product was stored at 4 and 25° C. A significant drop in infectious titer was detected in cells infected with virus stored at 25° C. for 48 hours with respect to freshly formulated virus stored at −80° C. (3±0.2×10⁵ to 4.42±0.2×10⁴ RLU, FIG. 1A). This trend continued for 7 days as luciferase levels fell from 2.1±0.02×10⁵ to 1.7±0.02×10⁴ RLU while luciferase expression from same preparation stored at 4° C. remained constant at 2.8±0.13×10⁵ RLU throughout the same time period (FIG. 1B). In sharp contrast, the number of virus genomes present in cells infected with the same preparations stored at both 4 and 25° C. did not correlate with changes in transgene expression (FIGS. 1C and 1D). Thus, luciferase expression was identified as the more sensitive assay for assessment of the amount of active virus present within the thin film matrix for optimization and long-term stability studies.

### Example 2—Impact of Film Components on Recovery of Live AAV During the Film Forming Process

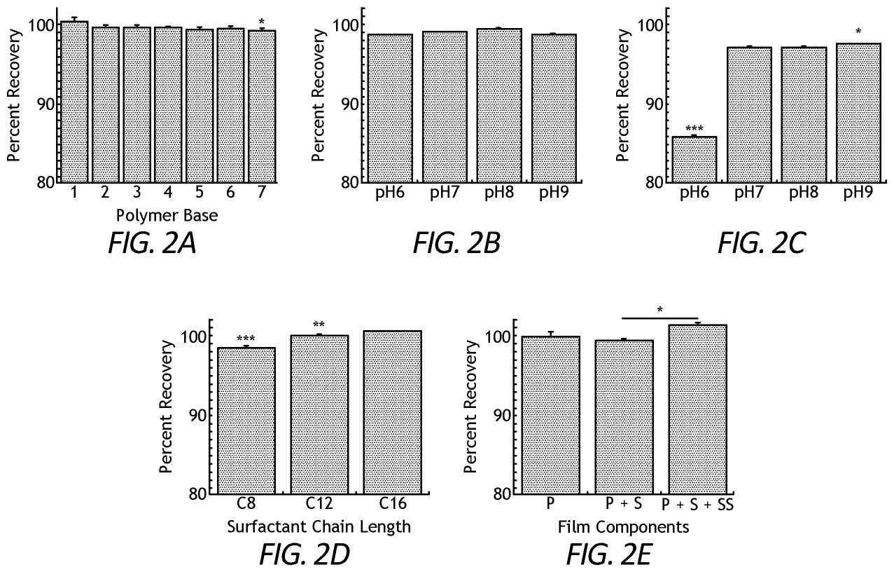

The formulation previously optimized for use with a recombinant adenovirus was highly viscous (4,000 cp). While this is acceptable for administration by the oral and nasal route, it is physiologically incompatible with intravenous administration, the manner by which many AAV-based and other vectors are currently administered. A series of seven different polymers with viscosities ranging from 15-4,000 cp were screened for their ability to maintain AAV infectious titer during the film forming process. The original infectious titer was retained by polymers with viscosities at or above 100 cp during the film forming process (Formulations 1-6, FIG. 2A). Films prepared from the lowest viscosity polymer (Formulation 7) were difficult to peel and as a result, demonstrated the most significant drop in infectious titer after drying (p<0.05). The polymers with the highest viscosity (Formulation 1, 4,000 cp) and lowest viscosity (Formulation 6, 100 cp) that maintained AAV viability were selected for additional study.

Changes in environmental pH can significantly impact the recovery of recombinant viruses during drying in a lyophilized or thin film platform. Films prepared with polymer in buffer of pH 8 offered a significant improvement in preserving AAV titer during the film forming process with respect to those prepared in buffers of pH 6, 7 and 9 (p<0.01, FIG. 2B). Storage of films prepared with each buffer at room temperature for 14 days revealed the impact of pH on long term stability as films prepared with polymer base in buffer of pH 6 retained 85.8±0.1% of the original titer while ˜97% of the original titer was retained in formulations prepared with base polymer alone in buffers of pH 7-9 (FIG. 2C). Surfactants are often included in film-based formulations to improve dispersion of a medicinal agent throughout the matrix, release of agent during dissolution and to improve the thermostability profile of live viruses at ambient temperatures. Prior work demonstrated that an amphipathic surfactant with a 16 carbon side chain preserved adenovirus infectivity during the film forming process while those with shorter side chains did not. This also seemed to be the case with the AAV9 vector as the same compound fully preserved AAV potency during the drying process while a compound containing an eight carbon side chain maintained 98±0.05% of the original titer (FIG. 2D). Inclusion of the surfactant in the film matrix with a sugar improved recovery of active virus from 98% (base alone, P) to 100% (P+S+SS, FIG. 2E). Taken together, formulations containing either high or low viscosity polymer base prepared at pH 8 with surfactant and sugar in the matrix were utilized for additional studies (e.g., studies described in Examples 3-8).

### Example 3—Impact of Formulation on AAV9 Release Characteristics from the Film Matrix

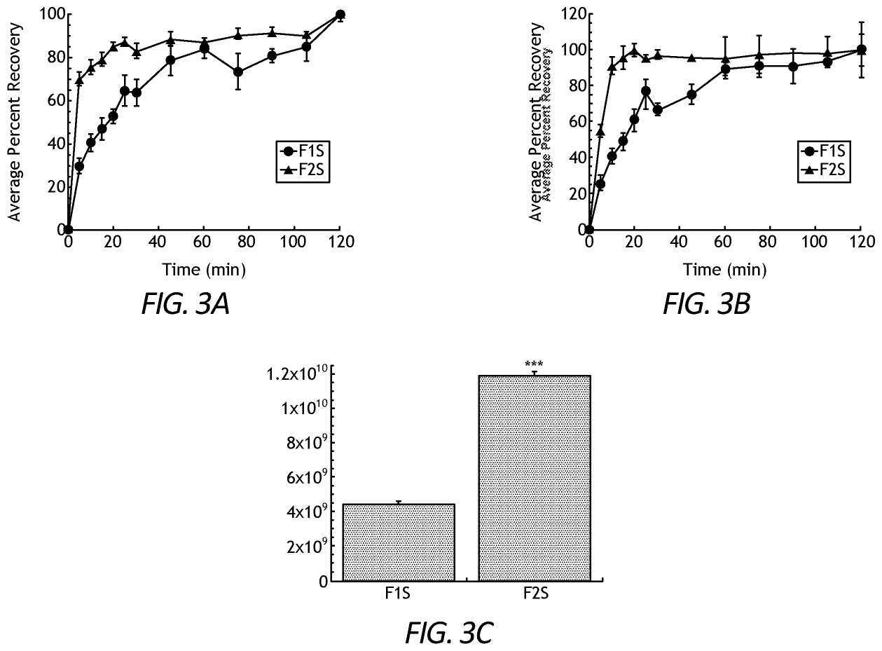

The release profile for the AAV9 vector from each formulation was characterized by assessing the transgene expression of live virus (FIG. 3A) and the number of virus genomes (FIG. 3B) in samples collected over a 2 hour period. Within 5 minutes, 70.3±3% of the total amount of infectious virus was released from the low viscosity film matrix (F2S) while only 29.7±3.5% of the total dose was released from high viscosity films (F1S) at the same timepoint (FIG. 3A). The composition of the F1S formulation was: 1.5% K4M HPMC, 2% sorbitol, 2% glycerol, 1% PMAL-C16, 10 mM Tris, pH 8.1. The composition of the F2S formulation was: 1.5% K100LV HPMC, 2% sorbitol, 2% glycerol, 1% PMAL-C16, 10 mM Tris, pH 8.1.

Approximately 80% of the total dose was released from the high viscosity film after 45 minutes while the same amount of virus was released from the low viscosity matrix within 15 minutes. A similar trend was observed for profiles generated performance of real time PCR on the same samples with 74.7±5.7% of the total number of virus genomes released within 45 minutes from the high viscosity matrix while 95.8±6.8% of the total dose was released from the low viscosity film within 15 minutes (FIG. 3B). Linear regression of each dissolution curve revealed that the low viscosity formulation released the AAV vector at a rate of 1.2±0.02×10¹⁰ vg/ml/min which was 3 times that observed from the high viscosity preparation (4.4±0.2×10⁹ vg/ml/minute, FIG. 3C).

### Example 4—Impact of Residual Moisture Content and Environmental Humidity on Recovery of Virus from Film Matrix

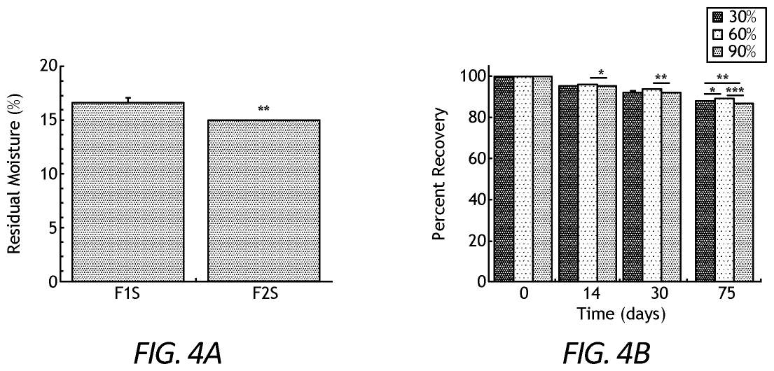

The amount of water that remains in a dried film dictates both the physical properties of the film and the thermostability profile of viruses in the dried state. Low viscosity films retained significantly less water than those prepared with the high viscosity polymer (14.9 vs 16.8%, FIG. 4A). In prior work with adenovirus, it was found that thermostability of virus at elevated temperatures was significantly influenced by the relative humidity of the environment in which films were stored. Thus, films prepared with the high viscosity formulation were packaged and stored in stability chambers set at 25° C. with varying levels of relative humidity (RH). After 14 days, at 90% RH infectious titer dropped by 4% and by 13% at day 75 (FIG. 4B). Films stored at 30% RH followed a similar trend. Films stored at 60% RH lost less than 10% of the initial dose of virus during the 75 day period, suggesting that all remaining long term stability studies be performed under these conditions.

### Example 5—Long-Term Stability and Resistance to External Stressors

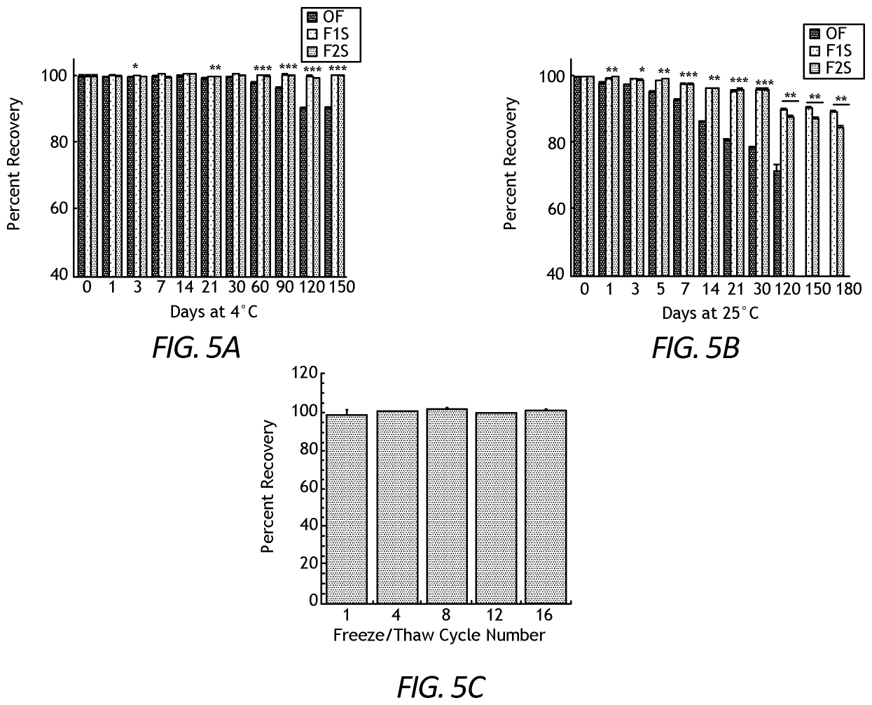

Films containing 1×10¹² virus genomes with either the high viscosity (F1S) or low viscosity (F2S) film matrix and stored in controlled environmental chambers held at 4° C./40-50% RH maintained 100% of their original titer for 150 days (FIG. 5A). Preparations containing the same concentration of virus stored in the original marketed liquid formulation displayed a significant drop of infectious titer to 97.7±0.1% after two months and gradually fell to 90.6±0.2% at the end of the study. When stored at 25° C., the infectious titer of the same preparation fell to 93±0.1% of the original titer by day 7, to 80.9±0.3% by day 21, 71.7±1.8% by day 120 and was undetectable after 5 months (FIG. 5B). There was no significant difference between the high and low viscosity formulations for the first 30 days at 25° C. with approximately 96% of the original dose found in films prepared from each formulation. After 150 days, the amount of infectious virus remaining in films prepared with the high viscosity formulation was significantly higher than that in those prepared with the low viscosity matrix (90.6±0.2% vs. 87.2±0.2%). By the end of the six month time period, the amount of infectious virus present in the low viscosity matrix dropped to 84.7±0.4% while that of the high viscosity material remained at 90%. Films prepared in the high viscosity matrix also fully preserved infectious virus during 16 freeze-thaw cycles that involved 24 hour alternating storage at −80 and 20° C. (FIG. 5C).

### Example 6—In Vivo Performance of AAV9 In Films: Short Term Storage at 4° C.

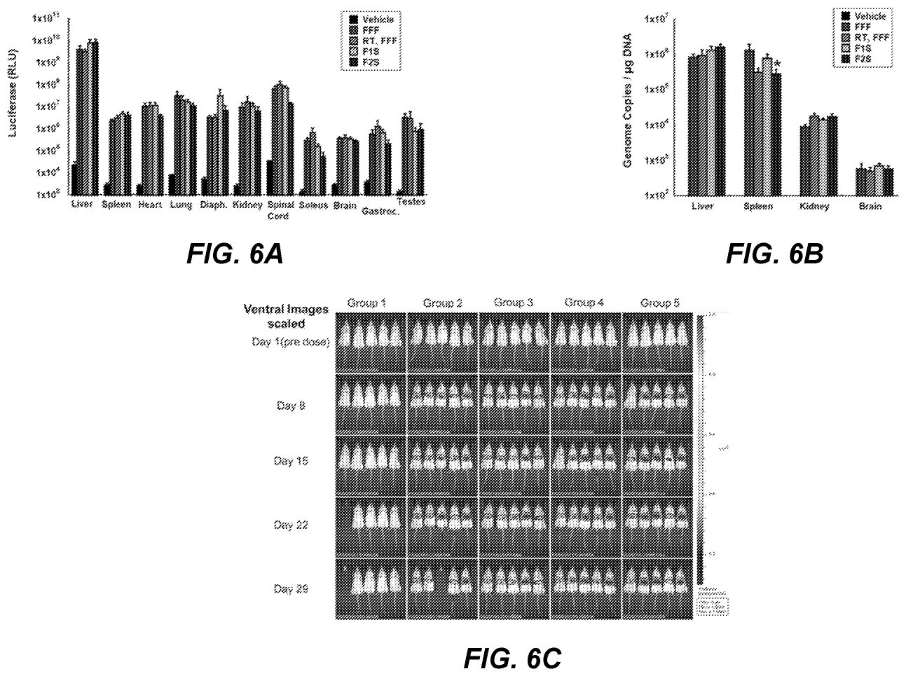

When virus stability within each film matrix at 4° C. seemed promising, an aliquot of films stored at 4° C. for 30 days were shipped without cool packs/dry ice from Austin, Texas to Research Triangle Park, NC. They were then rehydrated and administered at a dose of 1.5×10¹¹ vector genomes by tail vein injection to mice. Transgene expression was compared to groups given the same dose of freshly prepared virus stored frozen in the marketed formulation (FFF) and an aliquot of the same virus thawed and left at room temperature during the film forming process (temperature control, RT, FFF, FIGS. 6A-6C). Quantitative assessment of the luciferase transgene in organs collected 30 days after administration revealed that virus stabilized in both the F1S and F2S formulations transduced all organs to the same degree as freshly purified, frozen virus (FIG. 6A). Transgene expression in the liver was 2 logs higher than that in other organs. This was also evident during IVIS imaging during the course of the study where transgene expression peaked in the liver by day 22 (FIG. 6C). Analysis of key organs (liver, kidney, spleen and brain) by quantitative real time PCR revealed that the biodistribution of virus genomes was in line with transgene expression except for the spleen where the number of genome copies present in samples isolated from mice given virus in the F2S formulation was significantly lower than samples from mice given the fresh, frozen virus (2.8±1.0×10⁵ vg/μg DNA vs. 1.3±0.6×10⁶ vg/μg DNA, p<0.05, FIG. 6B).

### Example 7—In Vivo Performance of AAV9 In Films: Long Term Storage at 4° C.

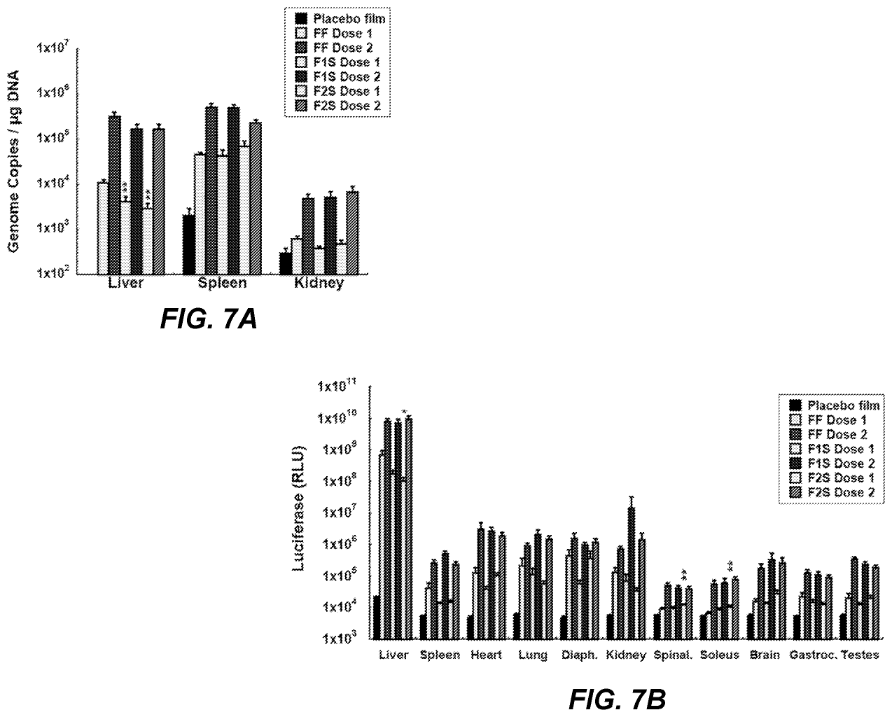

In a follow up study to the studies described in Example 6, AAV stored in each film formulation at 4° C. for 150 days was shipped without cool packs/dry ice from Austin, Texas to Research Triangle Park, NC. Films were rehydrated and virus administered by tail vein injection to mice at two different doses (1×10¹⁰ vg, Dose 1 and 1×10¹¹ vg, Dose 2, FIG. 7) to determine a dose effect and minimize potential saturation of the luciferase transgene in target tissues like the liver. As in the previous study, quantitative assessment of the luciferase transgene in organs collected 30 days after administration revealed that there was no significant difference in transgene expression achieved in all tissues of animals given freshly purified virus and that stabilized in either film formulation at the high dose (Dose 2, FIG. 7A). A significant reduction in transgene expression in the spinal cord (p=0.009) and the soleus (p=0.006) was noted in mice given the low dose of virus stabilized in the F2S formulation. Livers from mice given the lowest dose of virus (Dose 1) in either film formulation contained a significantly lower number of virus genomes than those from mice given the same dose of fresh, frozen virus (FF, FIG. 7B). This only resulted in a significant reduction in hepatic transgene expression in mice given the F2S formulation (p<0.05, FIG. 7A).

### Example 8—In Vivo Performance of AAV9 In Films: Long Term Storage at 25° C.

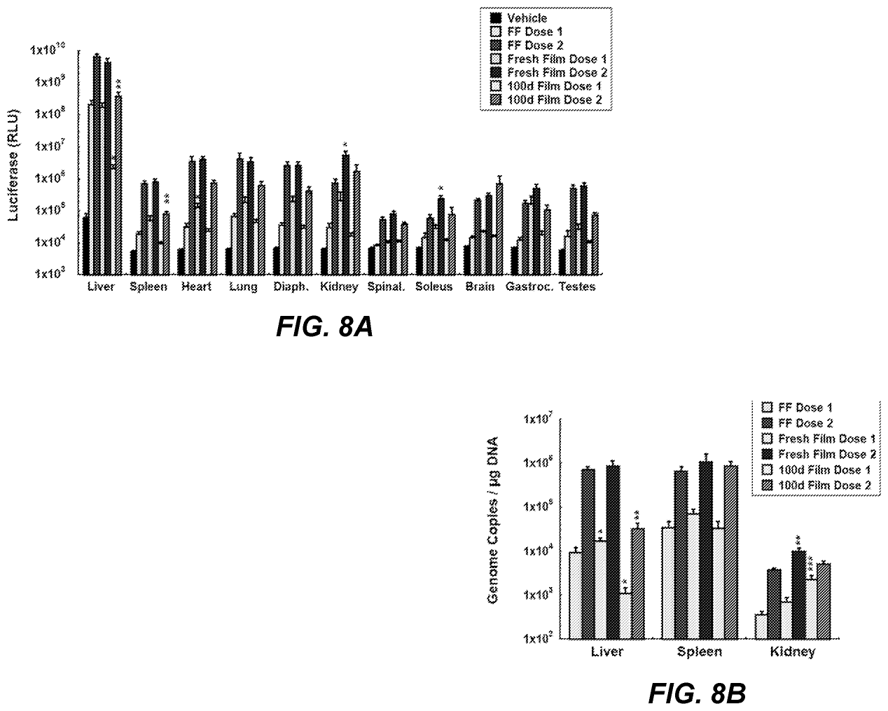

In a final proof-in-principle study, the transduction efficiency of AAV stabilized in films prepared with the F1S formulation and stored at 25° C. for 100 days was compared to that of films freshly prepared as well as fresh frozen virus (FIG. 8). Films were rehydrated and virus administered by tail vein injection to mice at two different doses (1×10¹⁰ vg, Dose 1 and 1×10¹¹ vg, Dose 2). The F1S formulation was selected for this study due to its superior performance with respect to the F2S formulation when stored at 25° C. (FIG. 8B). Quantitative assessment of the luciferase transgene in organs collected 30 days after administration revealed that there was no significant difference in transgene expression in tissues from animals given virus prepared in freshly made films and fresh, frozen stock at both doses (FF, FIG. 8A). An exception to this was that there was significantly more luciferase expression in the kidney (5.36±1.96×10⁶ RLU) and soleus (2.41±0.63×10⁵ RLU) after administration of the highest dose (Dose 2) with respect to that observed in kidneys ((7.47±2.21×10⁵ RLU) and soleus (6.06±1.60×10⁴RLU)) from mice given fresh, frozen virus. The number of virus genomes isolated from the kidneys of each treatment group followed the same trend (9.95±1.9×10³ vg/μg DNA, fresh film vs. 3.74±0.43×10³ vg/μg DNA, fresh frozen, FIG. 8B). Transgene expression in tissues obtained from animals given virus prepared in films stored at 25° C. for 100 days was slightly reduced or remained unchanged for most tissues with respect to those obtained from mice given, fresh frozen virus at each dose tested (FIG. 8A). However, a 2 log drop in RLU (p<0.05) was noted in the liver of animals given the low dose of virus from 100 day films while those given the highest dose demonstrated a 1.2 log drop in RLU (p<0.01) with respect to those given fresh, frozen virus. The number of virus genomes in livers from these animals followed the same trend (FIG. 8B). While a significant decrease in transgene expression was also found in the spleens of animals given the high dose of virus from 100 day old films (p<0.01, FIG. 8A), the number of vector genomes present in these tissues were not statistically different from those given the same dose of fresh, frozen virus (FIG. 8B).

### Example 9—Studies of AAV-Containing Liquid Compositions

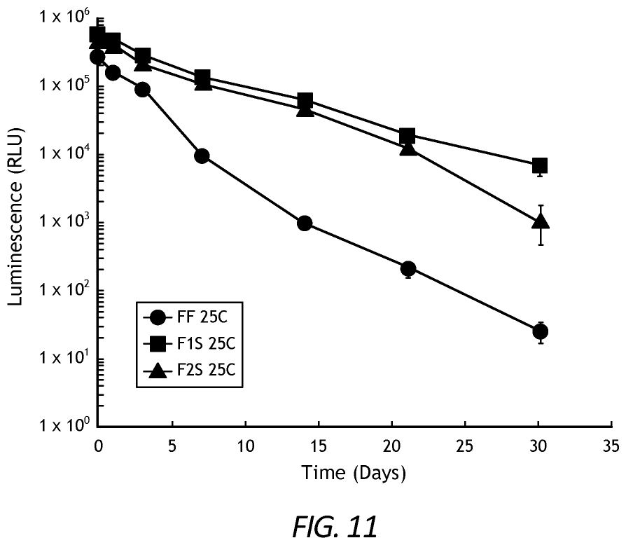

Virus containing the luciferase transgene was prepared in standard formulation (FF. phosphate buffered saline, 350 mM NaCl, 5% Sorbitol, 0.001% Pluronic F68 (pH 7.4), high viscosity film formulation (F1S, 1.5% HPMC K4M, 2% sorbitol, 1% PMAL, pH 6.5) or low viscosity film base (F2S, 1.5% HPMC K4M, 2% sorbitol, 1% PMAL, pH 6.5). Solutions were placed in sterile, sealed vials and samples taken over a period of 30 days. Infectious titer was determined by serial dilution, infection of HeLaRC32 cells and a standard luciferase assay. Results of these studies are shown in FIG. 11. These studies demonstrated that liquid film formulations provide superior physical stability profiles to AAV9 at room temperature (25° C./60% RH)

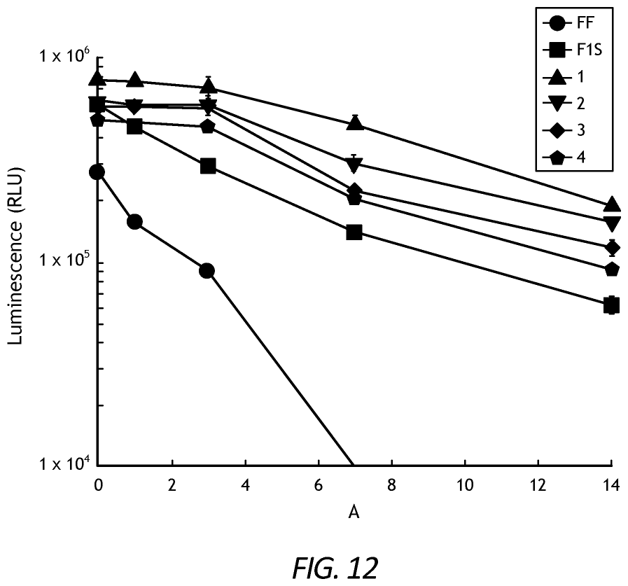

Virus containing the luciferase transgene was prepared in standard formulation (FF. phosphate buffered saline, 350 mM NaCl, 5% Sorbitol, 0.001% Pluronic F68 (pH 7.4), high viscosity film formulation (F1S, 1.5% HPMC K4M, 2% sorbitol, 1% PMAL, pH 6.5) or the F1S formulation with increasing amounts of a divalent cationic compound (1, lowest, 2 highest concentration). Solutions were placed in sterile, sealed vials and samples taken over a period of 14 days. Infectious titer was determined by serial dilution, infection of HeLaRC32 cells and a standard luciferase assay. Results of these studies are shown in FIG. 12. These studies demonstrated that cationic compounds support AAV9 stability at room temperature.

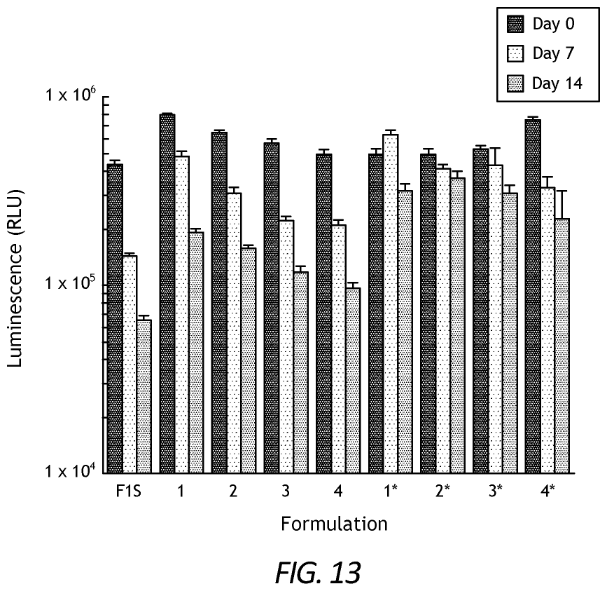

Virus containing the luciferase transgene was prepared in the F1S formulation with increasing amounts of a divalent cationic compound (1, lowest, 2 highest concentration) in the presence (Forms 1-4) or absence (Forms 1*-4*) of surfactant. Solutions were placed in sterile, sealed vials and samples taken over a period of 14 days. Infectious titer was determined by serial dilution, infection of HeLaRC32 cells and a standard luciferase assay. Results of these studies are shown in FIG. 13. These studies demonstrated that AAV9 stability at room temperature in liquid formulations improves in the absence of surfactant.

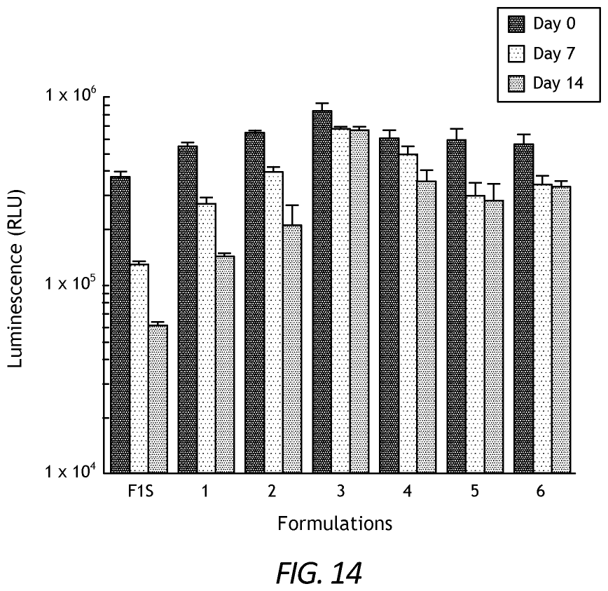

Virus containing the luciferase transgene was prepared in the F1S formulation without surfactant. Additional excipients were added to each preparation. Solutions were placed in sterile, sealed vials and samples taken over a period of 14 days. Infectious titer was determined by serial dilution, infection of HeLaRC32 cells and a standard luciferase assay. Results of these studies are shown in FIG. 14. These studies demonstrated that additional excipients improve AAV9 stability in liquid formulations at room temperature.

Virus containing the luciferase transgene was prepared in the F1S formulation without surfactant, Tris buffer pH 8.1. Additional excipients were added to each preparation. Solutions were placed in sterile, sealed vials and samples taken over a period of 14 days. Infectious titer was determined by serial dilution, infection of HeLaRC32 cells and a standard luciferase assay. Results from these studies are shown in Table 1. These studies demonstrated that formulations that maintain pH above 7.4 support AAV stability in the liquid form.

TABLE 1

| Formulation | Day 0 (pH) | Day 7 (pH) | Day 14 (pH) |
|---|---|---|---|
| F1S | 6.5 | 6.5 | 6.5 |
| 1 | 7.5 | 7.5-8 | 8.5 |
| 2 | 7.5 | 7.5 | 7.5 |
| 3 | 7.5 | 8.5 | 8.5 |
| 4 | 7.5 | 7.5 | 7.5 |
| 5 | 7.5 | 7.5 | 7.5 |
| 6 | 7.5 | 7.5 | 7.5 |

The formulations shown in FIGS. 12 and 13 are as follows:
F1S: 1.5% HPMC K4M, 2% sorbitol, 1% PMAL, pH 6.5 in Tris buffer (10 mM)
Formulation 1: F1S with 0.5 mM MgCl₂.
Formulation 2: F1S with 1.0 mM MgCl₂.
Formulation 3: F1S with 1.5 mM MgCl₂.
Formulation 4: F1S with 2.0 mM MgCl₂.

The formulations shown in FIG. 14 and Table 1 are as follows:
F1S: 1.5% HPMC K4M, 2% sorbitol, 1% PMAL, pH 6.5 in Tris buffer (10 mM)
Formulation 1: F1S without PMAL and with 1 mM MgCl₂.
Formulation 2: F1S without PMAL and with 0.2% alpha cyclodextrin.
Formulation 3: F1S without PMAL and with 0.2% beta cyclodextrin.
Formulation 4: F1S without PMAL and with 0.2% methyl beta cyclodextrin.
Formulation 5: F1S without PMAL and with 0.2% hydroxy propyl beta cyclodextrin.
Formulation 6: F1S without PMAL and with 0.2% gamma cyclodextrin.

### Example 10—Studies of AAV-Containing Film Compositions

### Summary

An AAV9 vector capable of expressing firefly luciferase (AAV9-CBA-Luc) was mixed with film base formulation, poured into 1 ml molds and films formed under aseptic conditions. Films were peeled and packaged in individual particle free-bags with foil overlays and stored at room temperature under controlled relative humidity. A portion of the films were shipped from Texas to North Carolina via overnight courier in an envelope without ice or cold blocks for in vivo testing. Otherwise identical liquid versions of the film compositions (prior to film formation) containing the same concentration of viral vector, and also control preparations containing the same concentration of the viral vector in a currently used liquid formulation, were stored at room temperature and 4 and −80° C. for comparison.

Over 95% of the original infectious dose of AAV was recovered after drying as determined by in vitro transduction assays measuring transgene expression and internalized viral genome copies. In contrast, infectious virus particles could not be detected in the liquid formulation after 30 days at room temperature with large aggregates detected by dynamic light scattering at 14 days transitioning to subviral particulates at day 30. Stability profiles of the same preparation stored at 4° C. for 30 days were not statistically different from vector stored in film at room temperature at the same timepoint. Films taken from room temperature storage and placed at 40° C. with 20% relative humidity for 3 days demonstrated a loss of less than 5% of the original infectious titer. Initial mechanistic studies suggest that elements of the film matrix directly bind to capsid proteins to stabilize them and shield them against environmental stressors. In vivo studies in mice demonstrated that vector biodistribution and transgene expression profiles of vector dried within the film matrix were similar to those of vector stored frozen in validated formulation. These results indicated that storage of AAV vector in the novel formulations, in both dry film and liquid form, facilitates easy transport of vector to remote sites without compromising in vivo performance.

### Results

In order to investigate the possibility of generating a storage formulation for AAV vectors for later administration (e.g., injected), hydroxylpropyl methylcellulose (HPLC) of different molecular weight and chain length were used to generate the storage formulation HPMC 1.5%, Sorbitol 2%, Glycerol 2%, PMAL-C16 1%. The different HPMC used were: K4M (viscosity 4000 centipoise (cp)), A4M (viscosity 4000 cp), F4M (viscosity 4000 cp), A15C (viscosity 1200-1800 cp), A4C (viscosity 400 cp), K100LV (viscosity 100 cp), A15VL (viscosity 12-18 cp). Each of these containing the same amount of AAV9 with a luciferase reporter gene expression cassette (AAV-luc) was generated, in liquid and dry film form. AAV present in both forms were then tested for the ability to transform cells in culture. The formulations that initially contained the same amount of AAV were used to transform otherwise identical cells. The recipient cells were then analyzed for luminescence conferred by the AAV vectors to determine the transduction efficiency, and the results compared to determine any differential effects on the AAV vector contained within each formulation. The results indicated that all versions of HPMC resulted in formulations that produced similar transduction efficiencies of the AAV contained therein, indicating no appreciable effect of the different HPMCs on the AAV with respect to transduction efficiency under the tested conditions. Both liquid form and dry film form performed similarly. Neither of these forms underwent any appreciable storage time or were subject to temperature or atmospheric extremes (ambient temperature, ambient atmospheric pressure).

Further experiments were performed using the K4M (viscosity 4000 cp), referred to as F1S, or the K100LV (viscosity 100 cp), referred to as F2S, HPMC.

In order to study the physical stability of the AAV vector within the formulations, F1S and F2S containing AAV-luc in film form were stored at 4° C. or 25° C. at 60% relative humidity (RH) for varying lengths of time, and then tested for AAV transduction efficiency in vitro as in above. These were compared to buffered formulation typically used in AAV storage (control), and also F1S in liquid form (without film formation) identically stored. The film versions were rehydrated just prior to use in the cell culture assay. Results indicated that the F1S and F2S formulations, both liquid and dry, preserved the AAV vector when stored at 4° C., the formulations being significantly better than identical storage in the control buffered formulation.

Herein, viscosity is expressed in centipoise unit (cp). Centipoise is a centimeter-gram-second unit of viscosity, equal to 1/100 (0.01) poise.

Results were obtained with storage of the AAV vector in the various formulates at 25° C. for 0, 1, 3, 5, 7, 14, 21, or 30 days at room temperature. F1S and F2S both preserved the AAV vector considerably more than the control formulation at 25° C. even after 30 days, with considerable drop off in the control formulation occurring after day 3, then becoming almost undetectable by day 30. The stability of the AAV vector in the control formulation was studied over time at both 4° C. and 25° C. by similar in vitro assays as described above. When stored at 25° C., a substantial drop off in preservation of the AAV vector is seen in the control formulation starting at day 7, further decreasing considerably over time, with complete loss of detectable AAV by day 150. Storage at 4° C. preserves stability of the AAV vector fairly well for a longer period of time, with a slight drop off beginning after day 30, and continual decrease in stability over time. In contrast, both F1S and F2S formulations preserved more than about 80% of AAV virus when stored at 25° C. for up to 150 days.

Results were obtained from studies looking at luciferase expression and show a significant decline in the recovered AAV vector in the control formulation when stored at 25° C. begins after about 5 days, with recovered vector being undetectable after 120 days, whereas both FS1S and F2S in dry film form are able to preserve the majority of the AAV vector. Storage of the AAV in film form at 25° C. preserved the vector significantly more than storage in the control formulation at 25° C. after about 5 days, and significantly more than storage in the control formulation at 4° C. after about 60 days storage. A significant decline in the recovered AAV in the control formulation when stored at 4° C. began after about 30 days.

Similar studies were performed to investigate the effect of relative humidity (60%, 20%, or 95%) on the storage capabilities of the film formulations. Results indicated that HPMC effects the preservation at increasing relative humidity. In particular, high molecular weight HPMC that has a higher viscosity (e.g., 4000 cp at a concentration of 2% in water) has a weaker preservation effect at extreme relative humidities (RH) e.g., up to 95% RH compared to that of low molecular weight HPMC having low viscosity (e.g., 100 cp at a concentration of 2% in water).

In vivo studies were performed to further investigate any effects the formulations may have on the AAV transduction efficiency and biodistribution. Group 1 contained vehicle control only, which was saline with no AAV. Group 2, Control A was the AAV-luc in buffered control, frozen at −80° C., shipped on dry ice, then used. Group 3, Control B, was the AAV-luc in buffered control, frozen at −80° C., thawed and left at room temperature for approximately 8 hours, refrozen and shipped on dry ice. Groups 4 and 5 contained the AAF-luc within F1S or F2S formulations in dried film, stored at 25° C., for thirty days, shipped via overnight mail, prior to administration. Group 4, the F1S contained 10× the amount of AAV-luc within the same amount of the film, to investigate possible effects of dilution prior to administration. No toxicity was observed, as shown in FIG. 7. The different formulations and controls were administered to mice systemically (via tail vein injection), and AAV transduction determined by luciferase expression as detected by in vivo analysis. Results show that there was no significant difference in gene expression in Groups 2-5, indicating similar transduction efficiencies resulting from the preservation of the AAV vector by the film formulations at room temperature for long period of time (e.g., 30 days).

Further studies were done with respect to biodistribution of the AAV in various tissues of the recipient mice. Specific organs obtained from mice treated as describes above for Groups 1-5, were harvested and analyzed ex vivo for AAV transduction via luciferase expression therein. The results, indicated no specific effect of the formulations on tissue distribution from systemic administration.

Preservation of the AAV vector within the various formulations was also investigated by analyzing genomic copy number by quantitative PCR, and those results compared to results from similar experiments performed by luciferase analysis of expressed reporter cassette from stored AAV vector. Results presented in Tables 2 and 3, respectively, indicated that the genome copy number was preserved along the same lines as the transduction efficiency.

TABLE 2

| Time point | | | | |
|---|---|---|---|---|
| (days) | Buffer - 4 C. | Buffer - 25 C. | F1S-25 C. | F2S-25 C. |
| 0 | 100.00 | 100.00 | 100.00 | 100.00 |
| 1 | 104.75 | 94.52 | 98.01 | 98.12 |
| 3 | 104.75 | 94.52 | 98.01 | 98.12 |
| 5 | 102.06 | 79.43 | 94.28 | 94.15 |
| 7 | 99.88 | 60.8 | 91.54 | 91.37 |
| 14 | 100.64 | 44.84 | 89.81 | 89.9 |
| 21 | 98.83 | 38.69 | 89.07 | 89.55 |
| 30 | 98.72 | 32.58 | 60.57 | 54.57 |
| 60 | 76.01 | 18.21 | 83.18 | 79.14 |
| 120 | 68.93 | 0 | 83.99 | 80.53 |
| 150 | 104.75 | 94.52 | 98.01 | 98.12 |

TABLE 3

| | Calculated | Theoretical | |
|---|---|---|---|
| | copy | copy | |
| Label | number/ml | number/ml | Ratio |
| Control undialyzed 1e14 vp/ml | 5.84E+13 | 1.00E+14 | 1.713 |
| Fresh film 1.5e13 vp/ml | 2.65E+12 | 1.50E+13 | 5.655 |
| D106 RT film 1.5e13 vp/ml | 2.86E+12 | 1.50E+13 | 5.243 |
| Control undialyzed diluted 1e12 | 2.59E+11 | 1.00E+12 | 3.859 |
| vp/ml | | | |
| G2 - undialyzed control 1e10 | 1.29E+10 | 1.00E+10 | 0.773 |
| vp/ml | | | |
| G3 - undialyzed control 1e11 | 1.05E+11 | 1.00E+11 | 0.954 |
| vp/ml | | | |
| G4 - fresh film 1e10 vp/ml | 3.66E+10 | 1.00E+10 | 0.273 |
| GS - fresh film 1e11 vp/ml | 1.34E+11 | 1.00E+11 | 0.746 |
| G6 - D106 RT film 1e10 vp/ml | 2.93E+10 | 1.00E+10 | 0.341 |
| G7 - D106 RT film 1e11 vp/ml | 1.28E+11 | 1.00E+11 | 0.782 |
| G1 - blank film | <LOD | 0.0E+00 | — |

### Materials and Methods

Formulations. Formulations with AAV vector sample contained 1×10¹² vg/ml. Dried films contained the same amount prior to drying. Dry and liquid samples containing the same initial amount of virus were compared in the studies.

Film Reconstitution. Films were reconstituted according to the protocol shown on FIG. 16. Infectious titer of AAV vector embedded in the preparation was determined by infection of HeLa cells and visual tallying of cells indicating expression of the reporter cassette. Percent recovery was calculated using the following formula:

$$\% Recovery = [ \frac{log ( Infecfious Titer at t = l )}{log ( Infectious Titer at t = 0 )} ] \times 100$$

In Vivo Biodistribution. Whole body imaging was obtained using an IVIS machine. Transduction efficiency was determined after storage in the various formulations as indicated, by luciferase expression in live animals. C57BL/6 albino mice were administered the formulations in a single dose of 1.5×10¹¹ VG/animal (measurements prior to storage) in total volume of 150 μl via tail vein injection on day 1. The mice were then subject to whole body imaging over time, days 1, 8, 15, and 22. The mice were injected intraperitoneally with luciferin (10 mg/kg mouse), and living images were taken 10 minutes later to quantitatively detect light emissions representative of viral gene expression in an IVIS machine. Living Image Software was used to analyze the results.

Tissue Biodistribution. Ex vivo imaging of organs was done at days 30 and 31. Mice were sacrificed and subjected to whole body perfusions with 10 ml PBS. Whole organs were removed and incubated in luciferin for uptake, then imaged in an IVIS for quantitative detection of emitted light. Emissions were calculated using living Image Software.

All of the methods disclosed and claimed herein can be made and executed without undue experimentation in light of the present disclosure. While the compositions and methods of this invention have been described in terms of certain embodiments, it will be apparent to those of skill in the art that variations may be applied to the methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The referenced cited herein, to the extent that they provide exemplary procedural or other details supplementary to those set forth herein, are specifically incorporated herein by reference.

1. A composition comprising an adeno-associated virus (AAV) vector in a carrier comprising (a) a zwitterionic surfactant and (b) hydroxypropyl methylcellulose (HPMC).

2. The composition of claim 1, wherein the HPMC is between 0.5% and 3.0%.

3. The composition of claim 1, wherein the HPMC is about 1.5%.

4. The composition of claim 1, wherein the HPMC has a molecular weight (MW) that produces a viscosity that is less than 4000 cp at a concentration of 2% in water.

5. The composition of claim 1, wherein the HPMC is A4M, F4M, A15C, A4C, K100LV, E4M, E6LV, or A15LV.

6. The composition of claim 1, wherein the carrier further comprises a sugar.

7. The composition of claim 1, wherein the carrier comprises about 2.0% sorbitol.

8. The composition of claim 1, wherein the carrier comprises about 2.0% glycerol.

9. The composition of claim 1, wherein the carrier comprises between 0.1% and 5% of the zwitterionic surfactant.

10. The composition of claim 9, wherein the carrier comprises about 1% PMAL-C16.

11. The composition of claim 1, wherein the composition has a pH from 7.0 to 9.0.

12. The composition of claim 1, wherein the carrier comprises about 1.5% HPMC, about 2% glycerol, about 2% sorbitol, and about 1% PMAL-C16.

13. The composition of claim 1, wherein the composition is a liquid.

14. The composition of claim 1, wherein the composition is a substantially solid film.

15. The composition of claim 1, wherein the AAV vector is an AAV1 vector, an AAV2 vector, an AAV3 vector, an AAV4 vector, an AAV5 vector, an AAV6 vector, an AAV7 vector, an AAV8 vector, an AAV9 vector, an AAV10 vector, an AAV11 vector, an AAV12 vector, an AAV13 vector, or a hybrid thereof.

16. The composition of claim 15, wherein the AAV vector is an AAV9 vector.

17. A method for storing an AAV vector comprising formulating the AAV vector in a composition of claim 1.

18. The method of claim 17, wherein the method comprises storing the AAV vector in the composition for at least 14 days at a temperature of at least 0° C.

19. The method of claim 17, wherein the method comprises storing the AAV vector in the composition for at least 14 days at a temperature of at least 15° C.

20. The method of claim 17, wherein, after storing, the AAV vector is preserved by at least 80% as measured by infectivity, transduction efficiency, and/or vector genome copy.

21. A method of delivering an AAV vector to a subject, the method comprising administering to the subject an effective amount of the composition of claim 1.

22. The method of claim 21, wherein the composition is administered to the subject intravenously.

23. The method of claim 1, further comprising, prior to administering the composition to the subject, storing the composition for at least 14 days at a temperature of at least 0° C.

24. The method of claim 21, further comprising, prior to administering the composition to the subject, storing the composition for at least 14 days at a temperature of at least 15° C.

25. A method for making a stabilized AAV vector composition, the method comprising forming an aqueous solution comprising an AAV vector, a zwitterionic surfactant, and HPMC.

26. The method of claim 25, wherein the HPMC has a molecular weight (MW) that produces a viscosity that is less than 4000 cp at a concentration of 2% in water.

27. The method of claim 25, wherein the aqueous solution comprises about 1.5% HPMC, about 2% glycerol, about 2% sorbitol, and about 1% PMAL-C16, wherein the aqeuous solution has a pH between 7.0 and 9.0.

28. The method of claim 25, further comprising drying the aqueous solution to form a substantially solid film.

29. The method of claim 28, further comprising (a) storing the substantially solid film for at least 14 days at a temperature of at least 0° C.; and (b) dissolving the substantially solid film in an appropriately buffered aqueous solution.

30. A pharmaceutical composition comprising between 1×10⁶ to about 1×10¹⁶ vg/ml of an AAV vector formulated within:
(a) from about 0.1% to about 5% wt/vol hydroxypropyl methylcellulose (HPMC);
(b) from about 0.5% to about 5% glycerol;
(c) from about 0.5% to about 5% sorbitol; and
(d) from about 0.1% to about 5% PMAL-C16; and
wherein the pH of the composition is from about pH 6.0 to 9.0.

31. The pharmaceutical composition of claim 30, wherein the composition comprises 1.5% HPMC, 2% glycerol, 2% sorbitol, 1% PMAL-C16, and has a pH of between 7.0 and 9.0.

32. A composition comprising an agent in a substantially solid carrier comprising hydroxypropyl methylcellulose (HPMC) and a zwitterionic surfactant, wherein the HPMC has a molecular weight (MW) that produces a viscosity that is less than 4000 cp at a concentration of 2% in water, and wherein the composition has a pH from 7.0 to 9.0.

33. The composition of claim 32, wherein the HPMC has a MW that produces a viscosity that is less than 1800 cp at a concentration of 2% in water.

34. The composition of claim 32, wherein the agent is an adeno-associated virus (AAV) vector.

35. The composition of claim 32, wherein the agent is a polypeptide, small molecule, or nucleic acid.

36. The composition of claim 32, wherein the carrier comprises about 1.5% HPMC, about 2% glycerol, about 2% sorbitol, and about 1% PMAL-C16.
